(19) United States
(12) Patent Application Publication
(10) Pub. No.: US 2024/0219374 A1
(43) Pub. Date: Jul. 4, 2024

(54) A Triple Co-Culture System for Drug Screening of Patient-Derived Cancer Cells and Methods of Use

(71) Applicants: OncoPrecision Corporation, New York, NY (US); Consejo Nacional de Investigaciones Científicas y Técnicas (CONICET), Caba (AR)

(72) Inventors: Ramiro Gastón SORIA, Maplewood, NJ (US); María Candelaria LLORENS DE LOS RÍOS, Córdoba (AR); Gerardo Alberto GATTI, Córdoba (AR); Tarek Ali ZAKI, New York, NY (US)

(21) Appl. No.: 18/556,227

(22) PCT Filed: May 26, 2022
(86) PCT No.: PCT/US2022/031105

§ 371 Date: Oct. 19, 2023

Related U.S. Application Data
(60) Provisional application No. 63/194,051, filed on May 27, 2021.

(51) Int. Cl. G01N 33/50; C12N 5/09

(52) U.S. Cl. CPC G01N 33/5011; C12N 5/0693; C12N 2503/02; G01N 2500/10

(57) ABSTRACT

The present disclosure provides a triple co-culture of a patient-derived cancer cell (PDC), a non-tumor derived cell that supports growth of the PDC and, optionally, may serve as an internal normal cell control (a “Tox Control” or “TC” cell), and a tumor-derived cell that is not derived from the patient and that exhibits sensitivity to at least one anti-cancer drug or drug combination (a “System Control” cell). The present disclosure also provides methods of use such triple co-culture systems, in for example, ex vivo personalized medicine, drug-discovery, drug development, and pre-clinical validation of candidates for clinical trials.

### CROSS-REFERENCE TO RELATED APPLICATION

This application claims priority benefit to U.S. provisional application Ser. No. 63/194,051, filed May 27, 2021, which application is incorporated herein by reference in its entirety.

### INTRODUCTION

Several technical obstacles limit the ability to assess ex-vivo drug testing on primary, patient-derived cancer cells. For example, ex vivo cultures of such cancer cells rapidly lose viability after being removed from their native microenvironment. Also, the small amounts of viable cancer cells that may be obtained from a biopsy limits screening of large collections of drugs or drug combinations. In addition, the mechanism of action of many drugs requires the active proliferation of cells to trigger the cytotoxic phenotype which, even under optimal growing conditions, may take several days. Such variability is not considered in multiple assays, which usually employ cell viability or cell death readouts within the first 48 to 72 hours after treatment. On the other hand, performing long ex vivo experiments (1 week or longer) produce high cellular confluence, which can complicate the quantification of cell viability through bulk methods such as MTT or Cell Titer Glo (CTG) assays. Attempts to address these variables have shown limited success.

Methods and systems are desired for cancer drug screening that address the problems with the conventional techniques and facilitate drug screening of cancer cells from patients.

### SUMMARY

The present disclosure provides a triple co-culture composition of a patient-derived cancer cell (PDC), a non-tumor derived cell that supports growth of the PDC and, optionally, may serve as an internal normal cell control (a “Tox Control” or “TC” cell), and a tumor-derived cell that is not derived from the patient and that exhibits sensitivity to at least one anti-cancer drug or drug combination (a “System Control” cell or “SC” cell). The present disclosure also provides methods of use of such systems in, for example, for ex vivo personalized medicine, drug-discovery, drug development, and pre-clinical validation of candidates for clinical trials. The triple co-cultures and methods described herein could be used for developing personalized therapies for cancer patients as well as developing new cancer therapies.

In some embodiments, the present disclosure provides a triple co-culture composition comprising: a first cell, wherein the first cell is a patient-derived cancer cell; a second cell, wherein the second cell is a non-tumor derived cell that supports growth and/or viability of the first cell; and a third cell, wherein the third cell is a tumor-derived cell that is not derived from the patient and that exhibits sensitivity to at least one anti-cancer drug or drug combination.

In some embodiments, the first cell and the third cell are derived from the same type of cancer. In some embodiments, the second cell and the third cell are non-isogenic. In some embodiments, the first cell, second cell, and the third cell are human cells.

The second cell can be of a non-tumoral cell type that is present in a tumor microenvironment of the first cell. For example, the second cell is a bone marrow stromal cell and the first cell is a PDC of hematological origin. Also, the second cell can be a fibroblast and the first cell can be a PDC from a solid tumor.

In some embodiments, at least one of the second cell and the third cell comprises a heterologous gene. For example, wherein the second cell is labeled with a first reporter. In some cases, the third cell is labeled with a second reporter. The first reporter and the second reporter can be the same or different from each other. In some embodiments, at least one of the first reporter and the second reporter is a fluorescent protein.

In some embodiments, the second cell and third cell promote growth of the first cell in the triple co-culture. In some cases, the first cell is a blood cancer cell, such as a leukemia. The first cell can also be a cell from a solid tumor, such as breast cancer.

Further embodiments of the invention provide a method for detecting a response of a patient-derived cancer cell to an anti-cancer therapeutic comprising: culturing the triple co-culture comprising a first cell, a second cell, and third cell as described above in the presence of a test agent, wherein the test agent is an anti-cancer therapeutic or a candidate anti-cancer therapeutic; and detecting the response to the test agent of the first cell, the second cell, and the third cell.

In some cases, the method comprises culturing the triple co-culture for a first time period; administering to the co-culture the test agent at the conclusion of the first time period; and detecting the response to the test agent, of the first cell, the second cell, and the third cell after a second time period. In some cases, the second period comprises at least one cell cycle of the first cell.

In some cases, the method further comprises comparing the response to the test agent of the first cell to the response to the test agent of the second cell and the third cell. In some cases, the response of the cells comprises changes in cell growth, cell number, cell size, and/or cell morphology.

In some embodiments, the method comprises scoring the first cell as sensitive to the test agent if the first cell exhibits an adverse response after the second time period, and the second cell does not exhibit an adverse response after the second time period. In some cases, the method comprises scoring the first cell as sensitive if the third cell exhibits an adverse response after the second time period. The adverse response can be apoptosis, necrosis, an inhibition of cell growth, an inhibition of cell division, or an abnormal cell morphology.

In some embodiments, the second cell comprises a first reporter and the third cell comprises a second reporter, and detecting the adverse response comprises detecting a loss of reporter activity. In some embodiments, detecting the response to the test agent comprises fluorescent activated cell sorting (FACS). FACS can be conducted at the conclusion of the second time period.

In some embodiments, the invention provides a method for analyzing a set of test agents comprising: culturing a set of triple co-cultures, each triple co-culture comprising a first cell, a second cell, and third cell as described above, wherein each triple co-culture in the set comprises the same patient derived cancer cell, and the same second cell and third cell; administering to each triple co-culture of the set a different test agent from the set of test agents or a different concentration of the same test agent; and detecting a response to the test agents of the first cell, the second cell, and the third cell in each triple co-culture.

In some embodiments, the method comprises: culturing each triple co-culture for a first time period; at the conclusion of the first time period, administering to each co-culture a test agent from the set of test agents or a different concentration of the same test agent; and detecting the response to the test agent of the first cell, the second cell and the third cell in each co-culture after a second time period. The second period can be at least one cell cycle of the first cell.

In some cases, the method comprises comparing the response to the test agent of the first cell to the response to the test agent of the second cell and the third cell. The response can be changes in cell growth, cell number, cell size, and/or cell morphology.

In some embodiments, the method comprises scoring the first cell as sensitive to the test agent if the first cell exhibits an adverse response after the second time period, and the second cell does not exhibit an adverse response after the second time period. In some embodiments, the method further comprises scoring the first cell as sensitive if the third cell exhibits an adverse response after the second time period. The adverse response can be apoptosis, necrosis, an inhibition of cell growth, an inhibition of cell division, or an abnormal cell morphology.

In some cases, the second cell comprises a first reporter and the third cell comprises a second reporter and detecting the adverse response comprises detecting a loss of reporter activity. Detecting the response to the test agent can be performed by FACS. In some embodiments, detecting the response to the test agent comprises conducting FACS at the conclusion of the second time period.

### BRIEF DESCRIPTION OF THE DRAWINGS

FIG. 1 provides a schematic overview of an example of the triple co-culture system.

FIG. 2 provides a schematic representation of different possible effects on the three types of cells in the triple co-culture.

FIG. 3 provides fluorescent activated cell sorting (FACS) plots obtained during production and isolation of “Tox control” (TC) cells. The TC cells were transduced with a lentivirus expressing a puromycin selection marker and fluorescent reporter protein (cayenne-RFP).

FIG. 4 provides fluorescent activated cell sorting (FACS) plots obtained during production and of isolation of “System control” (SC) cells. SC cells were transduced with a lentivirus expressing a puromycin selection marker and fluorescent reporter protein (dasher-GFP).

FIGS. 5A-5D provides the results of co-culture of HS-5 cells as TC cells and Jurkat cells as SC cells as well as cytometric analyses of these cells.

FIGS. 6A-6B provide the results of FACS analysis of PDC cells at various stages of isolation, purification, and post-thawing.

FIGS. 7A-7C: FIGS. 7A-7B provide the results of FACS analysis of PDC cells cultured with HS-5-conditioned medium, in dual co-culture with HS-5 TC cells or in triple co-culture with HS-5 TC cells and Jurkat SC cells. FIG. 7C provides the results of culturing PDC cells ex vivo for six days as a single suspension (mono-culture) in comparison to a triple co-culture system both in Acute Lymphoblastic Leukemia (ALL) and in Acute Myeloid Leukemia (AML).

FIGS. 8A-8C: FIG. 8A provides schematic representation of an example of a triple co-culture system and use to screen test agents. FIG. 8B provides an example of a timeline for producing a triple co-culture of TC cells, SC cells, and PDC cells, as well as treatment with a drug and analysis by flow cytometry. FIG. 8C provides a list of examples of drugs and combinations of drugs that can be screened in a Drug Activity and Resistance Test (DART) using triple co-culture systems in which the PDC cells are obtained from a patient having ALL or AML.

FIGS. 9A-9F: FIG. 9A describes different types of responses that can be observed using the triple co-culture system and PDC cells. FIG. 9B shows a full dose-response heatmap of a 96-well plate with a part of the treatment matrix (23 treatments) of a patient (OP #23) depicted in FIG. 9A. FIG. 9C shows that Drug A: Venetoclax exerts a selective activity against the PDC cells, and no cytotoxic activity on the TC cells. FIG. 9D shows that Drug B: a combination of Cytarabine and Daunorubicine shows an active and toxic response, strongly affecting the PDC cells and SC cells. FIGS. 9E-9F show drugs and the concentrations used in the exemplified screening assay.

FIGS. 10A-10G: FIG. 10A shows testing of PDC cells from two AML patients using the triple co-culture system. FIG. 10B shows drug-to-drug correlations for the patients depicted in FIG. 10A. FIG. 10C shows drug-to-drug correlations for the effects on the SC cells in the screening assay performed for the patients depicted in FIGS. 10A and 10B. FIGS. 10D-10G show the drugs and their respective concentrations used in estimating a therapeutic effect of cancer drugs for individualized treatment.

FIGS. 11A-11B show the effects of the top performing mono-drugs and drug combinations identified in the initial assay on the PDC cells from the selected patient.

FIGS. 12A-12B show an example of 3D tumoroid formation of PDC cells and testing of a potential cancer drugs on the 3D tumoroid. FIG. 12A shows an exemplary method of a 3D tumoroid formation containing PDC cells, SC cells, and TC cells. FIG. 12B shows examples of magnetically printed 3D patient-micro-avatars (PMAs) using PDCs from an AML patient.

FIG. 13 shows a schematic representation of a protocol to assemble 3D PMAs using samples from solid tumor biopsies.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a.” “an.” and “the” include plural referents unless the context clearly dictates otherwise. Thus, for example, reference to “the molecule” includes reference to one or more proteins, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

A “patient-derived cell” refers to a primary cell obtained from a patient, e.g., a human. A “patient-derived cancer cell” refers to primary cell obtained from a patient, e.g., a human, that is cancerous or suspected of being cancerous.

The terms “cancer” or “tumor” are used interchangeably herein to refer to an abnormal growth of tissue, and which may be benign, pre-malignant or malignant. In the context of the patient-derived cancer (PDC) cells, such cells are usually pre-malignant or malignant.

A “liquid tumor” refers to any tumor cell present in body fluid of a patient such as the blood or bone marrow.

A “semi-solid tumor” refers to an anatomical structure, such as a lymph node, in which tumor cells of a liquid tumor have accumulated (e.g., as in lymphoma).

A “solid tumor” refers to an abnormal mass of tissue that usually does not contain cysts or liquid areas. Different types of solid tumors are named for the tissue of origin of the primary tumor. “Circulating tumor cells” (CTCs) are a rare subset of cells found in the blood of patients with solid tumors, and which function as a seed for metastases. Cancer cells metastasize through the bloodstream either as single migratory CTCs or as multicellular groupings-CTC clusters.

“Test agent” as used herein refers to an anti-cancer drug or combination of anti-cancer drugs (or candidate anti-cancer drug or combination of anti-cancer drugs) amenable for screening in an assay described herein. Candidate drugs include agents for which anti-cancer activity against a selected cancer type may not be known. “Drugs” include, for example, small molecule drugs, antibodies, nucleic acids (e.g., siRNAs, etc.), gene targeting systems (e.g., TALENs, ZFNs, CRISPR gene editing systems), and the like.

The term “cell culture” or “culturing of cells” refers to maintaining, propagating, and/or passaging cells in an in vitro environment. Cells can be in any arrangement such as individual cells, monolayers, suspensions, and/or cell clusters.

The term “operably linked” refers to functional linkage between molecules to provide a desired function. “Operably linked” in the context of nucleic acids refers to a functional linkage between nucleic acids to provide a desired function such as transcription, translation, and the like, e.g., a functional linkage between a nucleic acid expression control sequence (such as a promoter, signal sequence, or array of transcription factor binding sites) and a second polynucleotide, wherein the expression control sequence affects transcription and/or translation of the second polynucleotide.

The terms “patient” or “subject” as used interchangeably herein can refer to a human or to a non-human animal, e.g. a mammal, including humans, primates, domestic and farm animals, and zoo, sport, laboratory, or pet animals, such as horses, cows, dogs, cats, rodents, and the like.

“Genetic modification” or “genetically modified” as used herein refers to a cell into which exogenous nucleic acid (e.g., DNA or RNA, usually DNA) has been introduced to provide for a stable (e.g., permanent) genetic change. Where the nucleic acid is DNA, the exogenously introduced DNA may be integrated into the genome of the cell or may be maintained as an episomal element. A “stably genetically modified cell” generally refers to a genetically modified cell in which exogenously introduced DNA is inherited by daughter cells, which can be demonstrated by, e.g., the ability of the genetically modified cell to establish cell lines or clones that comprise a population of daughter cells containing the exogenously introduced DNA.

“Clone” or “clonal” refers to a cell, or population of cells, derived from a single common ancestor cell.

A “cell line” refers to a cell that, when cultured, provides a clonal population of cells of substantially uniform phenotype and genotype, which phenotype and genotype are stably and substantially maintained in in vitro culture for multiple generations, as well as for multiple passages (e.g., at least 5, 10, or 20 passages or more) in cell culture. “Cell line” as used herein usually refers to a eukaryotic cell line (e.g., mammalian cell, e.g., human cell).

The term “promoter” as used herein refers to transcriptional control sequences, such as enhancers, polyadenylation signals, and terminators that provide for and/or regulate transcription of a coding sequence of a gene encoding a protein, e.g., a reporter protein.

A “vector” or “expression vector” refers to a replicon, such as plasmid, phage, virus, artificial chromosome, or cosmid, in which a nucleic acid (e.g., gene) may be included so as to facilitate the introduction of the gene into, the replication of the gene in, and, in the case of an expression vector, expression of the gene, in a host cell.

An “expression cassette” refers to a nucleic acid containing a DNA encoding a gene product of interest (e.g., a reporter gene, a selection marker (e.g., drug resistance gene) operably linked to a promoter so as to provide for expression in a host cell.

### DETAILED DESCRIPTION

### Overview

The present disclosure generally provides a cell culture system that provides a suitable environment for the ex-vivo culture and drug sensitivity screening of primary patient-derived cancer cells (“PDCCs” or “PDC cells”). The cell culture system can provide a platform for a wide variety of uses, for example, supporting PDC cells for analysis (e.g., cytology, histology, immunobiology, growth rate, etc.), determining the sensitivity of the PDC cells to an anticancer treatment (which in turn can facilitate selection of a therapy for the patient from whom the PDC cells were obtained), assaying the effect of a candidate agent (e.g., in the context of a candidate drug screening assay), and/or identifying subjects suitable for a clinical trial. Certain embodiments of this disclosure provide a cell culture system, which is referred to herein as “triple co-culture”, “triple co-culture system”, or “triple co-culture cell system”. In general, in the triple co-culture systems described herein, PDC cells are co-cultured with at least two different cell types, and thus includes at least the following:
a first cell, wherein the first cell is a patient-derived cancer cell, as described above;
a second cell, wherein the second cell is a non-tumor derived cell that supports growth and/or viability of the patient-derived cancer cell; and
a third cell, wherein the third cell is a tumor-derived cell that is not derived from the patient and that exhibits sensitivity to at least one anti-cancer drug or drug combination.

One or both of the second and third cell can be optionally tagged, e.g., with a reporter protein, such as a fluorescent protein.

The second cell of the above triple co-culture system is generally referred to herein as a “Toxicity Control cell”, “Tox Control cell” or “TC cell” supports the growth and/or viability of the PDC cells in culture. In some embodiments, the TC cells also serve as an internal control in that TC cells can reflect sensitivity of normal cells of a test agent, e.g., cells having the same or similar anti-cancer drug toxicity as non-cancer cells found in the in situ microenvironment from which the PDC cells are derived. The third cell type in the triple co-culture compositions of the present disclosure, generally referred to herein as a “System Control cell” or “SC cell” provides an internal positive control for the test agent being assayed, e.g., the SC cells have a known sensitivity/resistance to the test agent being assayed. In certain cases, SC cells are cancer cells, usually cancer cell lines, that are of the same type of cancer as the PDC. The SC cells can optionally support the viability and/or growth of PDC cells, TC cells, or both.

In the methods disclosed herein, the triple co-cultured cells are treated with a test agent, which can be a drug, candidate drug, or combinations of drugs and/or of candidate drugs. The PDC cells, TC cells, and SC cells are evaluated, for example, by high-throughput flow cytometry, to determine the response, such as relative survival, of PDC cells, SC cells, and TC cells. The effect of a particular drug or drug combination on each of PDC cells, SC cells, and TC cells can be used to, for example, determine the suitability of the test agent as a therapy for the patient.

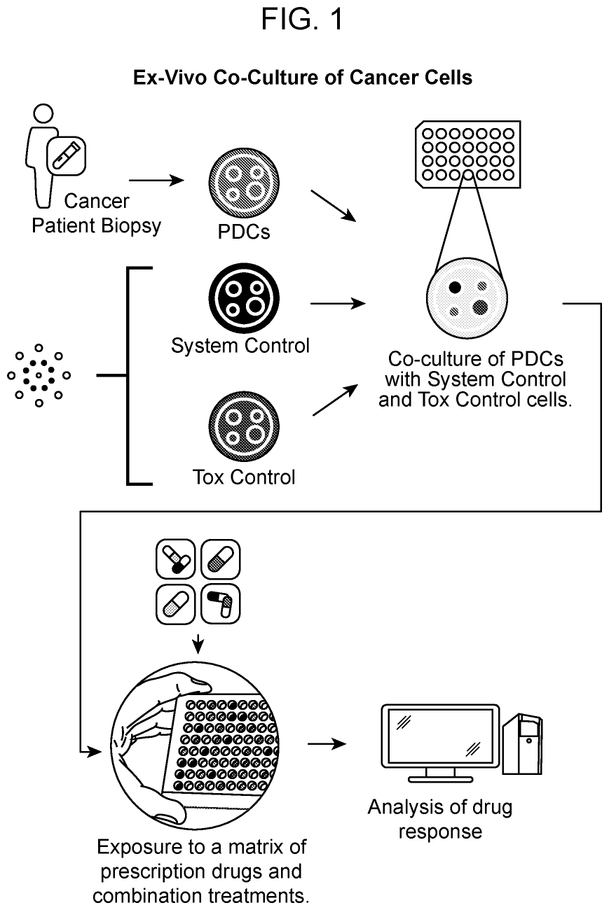

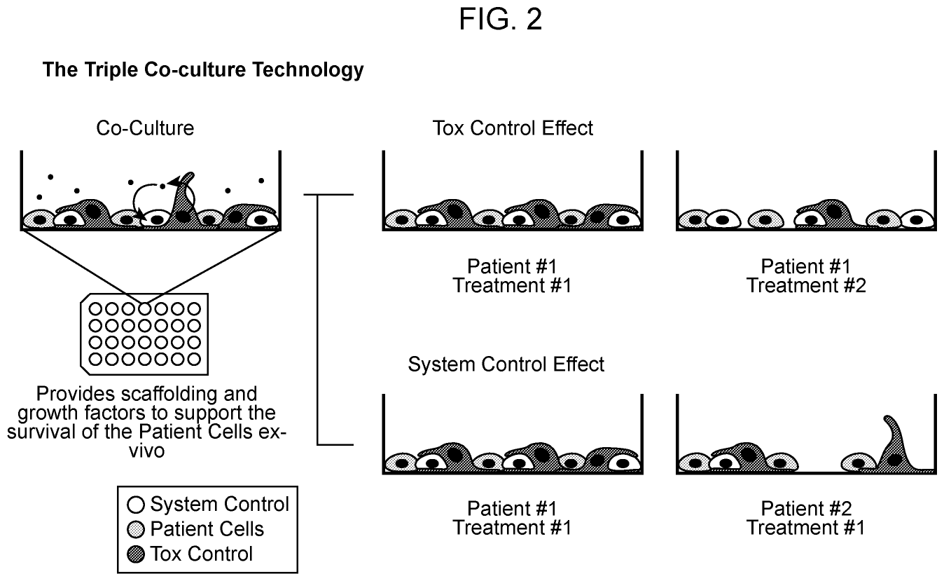

FIGS. 1 and 2 provide a schematic of an example of a triple co-culture system and use in a screen of prescription drugs.

As shown in FIG. 1, in certain embodiments of the invention, a biopsy containing cancer cells can be obtained from a patient. From such biopsy, PDC cells can be obtained by any suitable protocol, for example, using the methods of enriching or isolating cancer cells as described in this disclosure.

SC cells can be selected based on the type of cancer present in the patient. Particularly, SC cells can be selected so that they are of the same cancer type as present in the patient and of a known sensitivity to one or more anti-cancer drugs or drug combinations. The selected SC cells can be genetically modified to express a reporter protein and/or a marker protein to facilitate analysis of SC cells in the mixture of cells present in the triple co-culture. For example, SC cells can be genetically modified to express a fluorescent protein as a reporter protein. These SC cells can be readily identified and distinguished from the other cells in the triple co-culture because of the unique and detectable fluorescent protein.

Similarly, TC cells can be selected based on the type of cancer present in the patient. Particularly, TC cells can be selected so that they belong to a type of cells that supports growth and/or viability of the PDC cells in their natural environment. Such cells can be, for example, fibroblasts, adipocytes, bone marrow stromal cells, vascular endothelial cells, or pericytes. Selected TC cells can be genetically modified to express a reporter protein and/or a marker protein to facilitate analysis of TC cells in the mixture of cells present in the triple co-culture. For example, TC cells can be genetically modified to express a fluorescent protein as a reporter protein. The reporter and/or marker protein (e.g., fluorescent protein) expressed in TC cells is different from the reporter and/or marker protein (e.g., fluorescent protein) expressed in the SC cells. Accordingly, TC cells can be readily identified and distinguished from the other cells in the triple co-culture, e.g., due to their unique and detectable fluorescent protein.

The triple co-culture composition comprising a co-culture of PDC cells, TC cells, and SC cells can be established using any suitable protocol, for example, via sequential assembly of the triple co-culture as described below. Once the triple co-culture is established, it can be cultured in the presence of a candidate drug or a drug combination or a known drug or a drug combination. For example, as illustrated in FIG. 1, the triple co-culture can be exposed to a matrix of approved (e.g., prescription) drugs and/or a combination of such drugs. After appropriate incubation period in the presence of the drug or drug combination, the effects of the drug or drug combination on the PDC cells, TC cells, and SC cells is analyzed to determine the suitability of the tested drug or drug combination for treating the cancer in the patient.

An optimal drug or drug combination shows cytotoxic effects on the PDC cells while having no effect on the TC cells. The drug or drug combination may or may not show any effect on the SC cells; however, a positive control would be run with a drug or drug combination known to be cytotoxic on the SC cells. Such positive control would exhibit cytotoxic effects on the SC cells thereby indicating that the tested culture system is operating as desired.

FIG. 2 provides a schematic of a method for identifying a suitable treatment for a cancer in a patient. As shown in FIG. 2, two different triple co-cultures can be established with PDC cells from two patients (“Patient #1” and “Patient #2”) along with SC cells and TC cells. The triple co-cultures can be subjected to two different cancer treatments (“Treatment #1” and “Treatment #2”).

In the case of patient 1 with treatment 1 (schematically shown in the plates labeled Patient #1, Treatment #1), no cytotoxic effects are observed on TC cells, SC cells, or PDC cells. Therefore, while treatment 1 would not exhibit side effects, it would likely not be effective in treating the cancer in patient 1.

In the treatment of patient 1 with treatment 2 (schematically shown in the plate labeled Patient #1, Treatment #2), cytotoxic effects are observed on TC cells but not on SC cells and PDC cells. Therefore, treatment 2 would exhibit side effects without providing any clinical benefit in treating the cancer in patient 1.

In the treatment of patient 2 with treatment 1 (schematically shown in the plate labeled Patient #2, Treatment #1), cytotoxic effects are observed on TC cells and PDC cells but not on SC cells. Therefore, treatment 1 would provide some efficacy in treating the cancer in Patient #2; however, such therapeutic efficacy would also be associated with at least some side effects as indicated by the cytotoxic effect on the TC cells.

Thus, the triple co-culture composition and methods disclosed herein can be used to test and select personalized cancer therapy for patients with using in vitro cultures and testing. Also, the triple co-culture can be used to screen candidate drugs or drug combinations for identifying effective drugs or drug combinations.

PDC cells, TC cells, and SC cells, as well as examples of other components of the triple co-culture system and methods of use of the triple co-culture system, are described below.

### Patient Derived Cancer Cell (PDC Cells)

As noted above, a “patient-derived cell” refers to a primary cell obtained from a patient, e.g., a human. A “patient-derived cancer cell” refers to primary cell obtained from a patient, e.g., a human, that is cancerous or suspected of being cancerous. Where the PDC cells are used in the assays of the present disclosure to facilitate selection of a cancer therapy for a patient, the PDC cells are from the same patient. In such single patient assays, serum from the patient can also be collected to provide for culturing of the triple co-culture system with human autologous serum.

“Primary cells” are cells obtained directly from living tissue (e.g. biopsy material) and, following optional storage under suitable conditions (e.g., frozen, e.g., in liquid nitrogen using conventional methods), are established for growth in vitro. In general, primary cells have undergone very few population doublings in culture, and thus are therefore more representative of the main functional component of the tissue from which they are derived in comparison to continuous cell lines, which are naturally or artificially immortalized to facilitate passage and growth in cell culture. Primary cells are thus more representative of the in vivo state of a patient cell than are cell lines.

PDC cells can be obtained from any suitable cancer biopsy from a patient. The cancer can be a liquid tumor, a semi-solid tumor, or a solid tumor.

### Liquid and Semi-Solid Tumors

Liquid tumors are cancers present in body fluids, such as the blood or bone marrow. Hematologic cancers, such as lymphomas, leukemias and myelomas, are examples of liquid tumors. Involved lymph nodes containing accumulated cancerous cells of a liquid tumor are sometimes referred to in the art as a “semi-solid tumor”.

Certain non-limiting examples of leukemias include acute lymphocytic leukemia (ALL), acute myelogenous leukemia (AML), chronic lymphocytic leukemia (CLL), Chronic myelogenous leukemia (CML), Multiple Myeloma (MM) and other myeloproliferative disorders.

Certain non-limiting examples of lymphomas include Non-Hodgkin's lymphoma (NHL), diffuse large B-cell lymphoma, T-cell lymphoma, Burkitt's lymphoma and Hodgkin's lymphoma.

PDC cells of a liquid tumor can be obtained from a patient sample of peripheral blood, bone marrow, or, where cells of a liquid tumor accumulate in lymph nodes, from a lymph node. PDC cells of liquid tumors can include, for example, peripheral blood mononuclear cells (PBMCs), including lymphocytes (T lymphocytes, B lymphocytes, and natural killer (NK) cells monocytes, and dendritic cells.

Methods for isolating and/or enriching PDC cells from such samples is well known in the art. For example, enrichment or isolation of lymphoma cells can be accomplished using ficoll gradient centrifugation of a blood or bone marrow sample to isolate peripheral blood mononuclear cells (PBMCs). With the ficoll gradient centrifugation, PBMCs are obtained that contain cancerous cells as well normal cells. These PBMCs can be used as a source of PDC cells in the triple co-culture disclosed herein.

PDC cells can be further enriched in a cell population or isolated using cancer cell specific markers. For example, antibodies that specifically bind to cancer cell specific markers can be conjugated to a solid support, such as magnetic beads. Cells from a bone marrow biopsy or peripheral blood can be contacted with such solid support thereby capturing the cancer cells. The unbound cells can be washed off to enrich or isolate the PDC cells.

A cancer cell marker can be selected based on the type of cancer present in the patient. For example, multiple myeloma cells over-express CD133 and, hence, antibodies against CD133 can be used to enrich or isolate PDC cells from a patient sample.

Alternatively, PDC cells can be enriched in a cell population by depleting non-cancerous cells, e.g., using cell specific markers for non-cancerous cells. For example, antibodies that specifically bind to cell specific markers that are present on non-cancerous can be conjugated to solid support, such as magnetic beads. Cells from a bone marrow biopsy or peripheral blood can be contacted with such solid support thereby capturing the normal cells. The unbound cells can be washed off and collected to enrich or isolate the PDC cells.

In some cases, PDC cells can be enriched or isolated using cell specific markers and flow cytometry, such as fluorescent activated cell sorting (FACS). In FACS methods, cells from a bone marrow biopsy or peripheral blood can be treated with fluorescent labeled antibodies against certain cell specific markers that are present mainly on cancer cells and/or other cell specific markers that are predominantly present on non-cancerous cells. FACS gating can be used to separate PDC cells that express certain cancer cell specific markers from non-cancerous cells that do not express those cancer cell specific markers. In some cases, a threshold of marker expression for cancer cells or non-cancerous cells is used to separate the cancerous cells from the non-cancerous cells to enrich or isolate PDC cells.

Any suitable protocols for FACS can be used, such as use of appropriate buffers, various fluorescent labeled antibodies, setting up of various gates, and separation of the cells expressing certain types of markers from the cells expressing certain other types of markers.

Depending on the type of cancer present in a patient, any conventional or commercially available method could be used to isolate PDC cells from a patient. For example, use of specific cell surface markers that are known in the art could be used for enrichment or isolation of PDC cells from a patient sample of a specific liquid tumor.

### Solid Tumors

A solid tumor can be acral lentiginous melanoma, adenocarcinoma, adenoma, anaplastic thyroid cancer, brain tumor, breast cancer, colon cancer, colorectal cancer, cutaneous T-cell lymphoma, epithelioid sarcoma, esophageal cancer, follicular lymphoma, gastrointestinal cancer, head and neck cancer, hepatocellular carcinoma, intraocular melanoma, melanoma, nodular melanoma, nonmelanoma skin cancer, non-small cell lung cancer, ovarian cancer, ovarian epithelial cancer, pancreatic cancer, or uveal melanoma.

PDC cells from a patient having a solid tumor can be obtained from a solid tumor biopsy. A solid tumor biopsy can be processed to produce a suspension of cells containing cancer and non-cancerous cells. Such processing to provide such cell suspensions can involve any of a variety of available methods, for example, mechanical fragmentation and/or enzymatic dissociation (for example, by collagenase II and/or DNase I treatment), and, optionally, lysing red blood cells using an RBC lysis buffer, such as ACK (Ammonium-Chloride-Potassium) lysis buffer. Cell suspensions containing obtained after processing a solid tumor biopsy can be further processed to deplete non-cancer cells and/or enrich for cancer cells that are to be the subject of the assay. Such processing can involve, for example, the embodiments described above for obtaining PDC cells from liquid tumors.

PDC cells from a solid tumor in a patient can also be isolated from peripheral blood. Circulating tumor cells (CTCs) shed from the primary solid tumor and circulate in the blood. Such CTCs may serve as a seed for metastases. These cells can be isolated or enriched from peripheral blood of a patient.

CTCs can be isolated from peripheral blood using immunoaffinity-based methods, such as using antibodies that are conjugated to solid support and that specifically bind to CTCs. Microfluidic devices containing antibodies that specifically bind to CTCs can also be used. Certain such methods of isolating CTCs are described by Banko et al. (2019), Journal of Hematology & Oncology. Vol. 12, Article number: 48, which is incorporated herein by reference in its entirety.

Further, in some instances, PDC cells from a solid tumor (or from a liquid tumor) can be isolated from ascites or fluid collected in the abdominal cavity. Typically, a catheter or syringe is used to drain ascite from a patient's abdominal cavity in a process called paracentesis. Certain cancers, such as ovarian, pancreatic, liver, and colon cancers, are more likely to cause ascites and, therefore, PDC cells from patients having these cancers could be isolated from ascitic fluid.

Cells obtained from a solid tumor biopsy, CTCs, or ascitic fluid containing cancer cells can be processed to isolate or enrich PDC cells. For example, PDC cells can be further enriched in a cell population or isolated using cancer cell specific markers using a variety of different methods. For example, antibodies that specifically bind to cancer cell specific markers can be conjugated to solid support, such as magnetic beads. Cells from a solid tumor sample can be contacted with such solid support thereby capturing the cancer cells. The unbound cells can be washed off to enrich or isolate the PDC cells.

A cancer cell marker can be selected based on the type of cancer present in the patient. For example, cells from a basal type of breast carcinoma express CD44⁺/CD24⁺ and, hence, antibodies against CD44 and CD24 could be used to enrich PDC cells from a patient having a basal type of breast carcinoma. Also, certain ovarian cancer cells exhibit a high glutathione content phenotype and, hence, monochlorobimane, which is a quantitative marker of cells exhibiting high glutathione can be used to enrich PDC cells from a patient having an ovarian cancer.

Alternatively, PDC cells can be enriched in a cell population by depleting non-cancerous cells, e.g., using cell specific markers for non-cancerous cells. For example, e-cadherin is an epithelial marker with decreased expression in malignant carcinomas, but that is highly expressed in normal epithelial tissue. Thus, binding agents, such as antibodies that specifically bind to cell specific markers, such as e-cadherin that are present on non-cancerous can be conjugated to solid support, such as magnetic beads. Cells from a solid tumor sample can be contacted with such solid support thereby capturing the normal cells. The unbound cells can be washed off and collected to enrich or isolate the PDC cells.

PDC cells can also be enriched or isolated using cell specific markers and FACS. In FACS methods, cells from a solid tumor biopsy, CTCs, ascitic fluid containing cancer cells, or even peripheral blood can be treated with fluorescent labeled antibodies against certain cell specific markers that are present mainly on cancer cells and/or certain other cell specific markers that are present mainly on non-cancerous cells. FACS gating can be used to separate PDC cells that express certain cancer cell specific markers from non-cancerous cells that do not express certain cancer cell specific markers. In some cases, a threshold of marker expression for cancer cells or non-cancerous cells is used to separate the cancerous cells from the non-cancerous cells to enrich or isolate PDC cells.

Any suitable protocols for FACS can be used, such as use of appropriate buffers, various fluorescent labeled antibodies, setting up of various gates, and separation of the cells expressing certain types of markers from the cells expressing certain other types of markers.

Depending on the type of cancer present in a patient, a person of ordinary skill in the art can determine appropriate methods, for example, use of specific cell surface markers, for enrichment or isolation of PDC cells from a patient having a specific solid tumor.

### Processing of Cell Populations to Prepare PDC Cells

In addition to the enrichment and/or isolation methods described above, cell populations obtained from a patient can be subjected to additional processing prior to use inf a triple co-culture system of the present disclosure.

For example, patients' cells obtained from a tumor sample (e.g., liquid tumor, semisolid tumor or solid tumor) can be assayed for cell viability and, where cell viability in the sample is below an acceptable threshold, enriched for live cells. Assays to assess cell viability are well known in the art and include, for example, survival assays using 7-aminoactinomycin D (7-AAD) (see, e.g., Zembruski, et al. (2012) Anal. Biochem. 429, 79-81). PDC cell populations having at least 80%, at least 85% or at least 90% viable cells are of particular interest for use in the triple co-cultures systems of the present disclosure.

Cell populations for use as PDC cells can also be assayed to determine the percentage of pathological cells (cancerous cells) in the cell population. In general, cell populations having at least 20% pathological cells are suitable for use as PDC cells. The percent of pathological cells can be assayed according to any of suitable methods known in the art, with methods involving staining cells using cancer cell-specific reagents (e.g., a detectably labeled antibody that specifically binds a cell surface marker indicative of a cancer cell) and assay methods (e.g., FACS).

PDC cells can be used shortly after isolation to prepare a triple co-culture system as described below (e.g., without long term storage), or can be stored by freezing according to methods well known in the art. PDC cells can be stored, e.g., in culture medium with autologous human serum or with FBS. Optionally the culture medium can be TC cell-conditioned medium, where the TC cell used to prepare the condition medium can be the TC cell to be used with the PDC cell in the triple co-culture system.

### Toxicity Control Cells

As noted above, a TC cell of the present disclosure is a non-tumor derived cell that supports growth and/or viability of the PDC cell. Furthermore, since TC cells are non-cancerous cells, such cells also serve as a toxicity control cell, e.g., a proxy for a normal, non-cancerous cell, to assess toxicity of a test agent for non-cancerous cells present in the microenvironment of the PDC cell. TC cells can be selected to be a non-cancerous cell or cell line that is derived from a cell type present in the tumor microenvironment from which the PDC cell was obtained.

In co-culture with PDC cells, a TC cell may structurally support growth and viability of PDC cells in adherent culture, serving as a scaffolding and/or as otherwise supporting cells. A TC cell may also support growth and viability of PDC cells via secretion of factors, such as growth factors and other secreted factors. Secreted factors from TC cells may be provide support to PDC cells in either suspension cultures or in adherent cultures.

The type of TC cell used in a particular triple co-culture thus depends on the cancer present in the patient from whom PDC cells are obtained. In certain embodiments, the cancer present in the patient is, and the PDC cells are obtained from, a liquid tumor. A TC cell can be for any liquid tumor described above in connection with the PDC cells. In some embodiments, the cancer present in the patient is a solid tumor. A TC cell can be for any solid tumor described above in connection with the PDC cells.

In an exemplary embodiment, the cancer present in the patient is a liquid tumor, such as a hematological malignancy, such as a leukemia, a lymphoma, or a myeloma. In co-culture of PDC cells derived from such liquid tumors, TC cells belonging derived from bone marrow stromal cells can be used. In certain embodiments, the TC cells used with such PDC cells are a bone marrow stromal cell line. Non-limiting examples of bone marrow stromal cell lines that could be used as TC cells include HS-5 and HS27A. In a specific embodiment, the bone marrow stromal cell line is HS-5.

In certain embodiments, the cancer present in the patient is a solid tumor, such as a breast cancer or an ovarian cancer. In a natural tumor environment of a solid tumor, cancer cells are supported by cell types, such as fibroblasts, vascular endothelial cells, pericytes, and adipocytes. In a co-culture of PDC cells derived from such a solid tumor, TC cells can be used that belongs to a cell type, such as fibroblast, epithelial cell, vascular endothelial cell, pericyte, and adipocyte.

In certain embodiments, a TC cell belongs to the type of supporting cells from the same organ that has a cancer in a patient. For example, in a co-culture of the breast cancer cells from a patient, a TC cell can be a breast fibroblast cell line, a breast endothelial cell line, a breast adipocyte cell line, or a breast pericyte cell line. Similarly, in a co-culture of the ovarian cancer cells from a patient, a TC cell can be an ovarian fibroblast cell line, an ovarian endothelial cell line, an ovarian adipocyte cell line, or an ovarian pericyte cell line.

The following table (Table 1) provides examples of cancer types of PDC cells and corresponding cell lines that can be used as a TC cell.

| Cell line | Tissue of origin | Cell type |
|---|---|---|
| BJ-5ta | Foreskin | Fibroblast |
| hTERT-immortalized | skin | Endothelial cells |
| Dermal Melanocytes | | |
| TIME | Foreskin | Endothelial cells |
| hTERT-HPNE | Pancreas | Intermediary cells |
| | | formed during acinar- |
| | | to-ductal metaplasia |
| CHON-002 | Long bone; cartilage | Fibroblast |
| Human Primary CAFs | Multiple sites | Fibroblast |
| immortalized by hTERT | (i.e. breast, colorectal, | |
| | melanoma, ovarian, | |
| | pancreatic) | |
| MRC-5 | Lung | Fibroblast |
| hTERT lung fibroblast | Lung | Fibroblast |
| ASC52telo | Adipose tissue | hTERT-immortalized |
| | | adipose-derived |
| | | mesenchymal stem |
| | | cells (MSC) |
| HS-5 | bone marrow/stroma | Fibroblast-like |
| | | HPV-16 E6/E7 |
| | | transformed |
| HS-27 | bone marrow/stroma | Fibroblast-like |
| | | HPV-16 E6/E7 |
| | | transformed |
| WI-38 | Lung | Fibroblast |

TC cells can be genetically modified a detectable marker (“tag”) to facilitate analysis. Such detectable markers include a reporter protein, such as a fluorescent protein. As discussed below, SC cells can also optionally be genetically modified to express a selection marker, such as drug resistance marker, with the proviso that the drug resistance is to a drug different than that of the test agent(s) to be screened in the assay using the triple co-culture system.

The genetic modification of a TC cell can comprise stable transfection of a TC cell with a vector that encodes the reporter protein. In a stable transfection, the gene that encodes the reporter protein is incorporated into the genome of the cell and is stably expressed in the cells over generations.

Typically, an expression cassette comprising a nucleic acid encoding a reporter protein operably linked to a promoter is introduced into a cell via transfection. Promoter driving the expression of a reporter protein ensures that the reporter protein is expressed at a desirable level. Non-limiting examples of promoters suitable for use in eukaryotic cells include EF1α, those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is within the level of ordinary skill in the art. The expression vector may also contain a ribosome binding site for translation initiation and a transcription terminator.

A promoter can be a constitutively active promoter, i.e., a promoter that is constitutively in an active/“ON” state, or it can be an inducible promoter, i.e., a promoter whose state, active/“ON” or inactive/“OFF”, is controlled by an external stimulus, e.g., the presence of a particular temperature, compound, or protein.

A nucleic acid encoding a reporter protein can be introduced into a host cell by any suitable method, using any of a variety of expression vectors. Methods of introducing a nucleic acid into a host cell include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery.

In certain embodiments, the reporter protein in a TC cell can be specifically detected in the TC cells from the live co-culture. The specific reporter protein is selected such that it is not present in the other cells cultured in the triple co-culture so as to facilitate distinguishing the TC cells from the other cells in the culture.

In one embodiment, the reporter protein present in the TC cell is a fluorescent protein. Non-limiting examples of fluorescent proteins that can be used as a reporter protein in a TC cell include green fluorescent protein, red fluorescent protein, yellow fluorescent protein, blue fluorescent protein, or orange fluorescent protein. Examples of such fluorescent proteins include, but are not limited to: green fluorescent proteins such as, e.g., GFP (wt), Dasher, EGFP, Emerald, Superfolder GFP, Azami Green, mWasabi, TagGFP, TurboGFP, AcGFP, ZsGreen, T-Sapphire; blue fluorescent proteins such as, e.g., EBFP, EBFP2, Azurite, mTagBFP; cyan fluorescent proteins, such as, e.g., ECFP, mECFP, Cerulean, mTurquoise, CyPet, AmCyanl, Midori-Ishi Cyan, TagCFP, mTFP1 (Teal); yellow fluorescent proteins such as, e.g., EYFP, Topaz, Venus, mCitrine, YPet, TagYFP, PhiYFP, ZsYellowl, mBanana; orange fluorescent proteins, such as, e.g., Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, mTangerine; and red fluorescent proteins, such as Cayenne, mRuby, mApple, mStrawberry, AsRed2, mRFP1, JRed, mCherry, HcRedl, mRaspberry, dKeima-Tandem, HcRed-Tandem, mPlum, and AQ143.

A TC cell can also be genetically modified to express, in addition to a detectable marker, a selection marker, such as a drug resistance gene, with the proviso that the drug resistance conferred is to a drug different than that of the test agent(s) to be screened in the assay using the triple co-culture system.

A selection marker can also be used to maintain expression pressure on the TC cell so that the TC cell keeps expressing the genes introduced into the TC cell. For example, a selection marker and the reporter protein can be present on the same expression vector so that in transfected TC cells, the cells retain the expression vector and continue to express the reporter protein. Such drug resistance selection markers can confer resistance to any of a variety of drugs, G418, geneticin, zeocin, puromycin, blasticidin, and hygromycin B.

In certain embodiments, a TC cell genetically modified to express a selection marker and a reporter protein is enriched or isolated using FACS. For example, an appropriate cell line can be transfected with one or more nucleic acids that encode an antibiotic selection marker and a fluorescent protein.

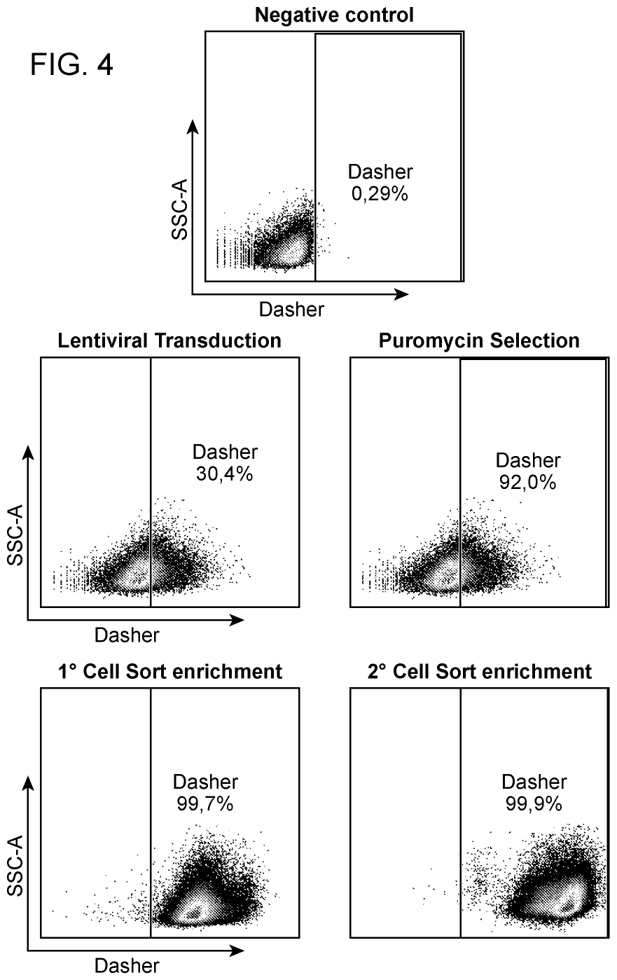

The cells expressing the antibiotic selection marker can then be subjected to FACS to separate cells that express the fluorescent protein from the cells that do not express the fluorescent protein. Thus, the TC cells that express both the antibiotic selection marker and the fluorescent protein can be isolated. An example of such isolation is described in FIG. 4.

TC cells can be used without long term storage, or can be stored by freezing according to methods well known in the art.

### System Control Cells

As noted above, a System Control (SC) cell of a triple co-culture system of the present disclosure is a tumor-derived cell that is not derived from the patient, and serves as an internal control for anti-cancer activity of a test agent. SC cells are thus cells that exhibit a known sensitivity to a test agent (i.e., anti-cancer agent or combination of anti-cancer agents (e.g., drugs)). The sensitivity of the SC cell to such anti-cancer drug or drug combination indicates proper operation of the triple co-culture. In the context of screening a candidate test agent (i.e., candidate anti-cancer agent or combinations of such (e.g., candidate anti-cancer drug(s)), an internal control for anti-cancer activity of the candidate test agent.

SC cells can be a cell of a cancer of a known test agent sensitivity or a cell line of a known test agent sensitivity, and generally is a cancer cell line of a known test agent sensitivity. Thus, the SC cell acts as in internal control for the anti-cancer effect (e.g., growth inhibition, e.g., killing) of an anti-cancer test agent at a known dose and/or period of exposure of the SC cell to the anti-cancer test agent, in culture.

The SC cell can be selected to be of the same type of cancer as the PDC cell in the triple co-culture. For example, in a triple co-culture where the PDC cells are breast cancer cells, the SC cell can also be a breast cancer cell, and can be of the same breast cancer cell type, e.g., with respect to genotype, e.g., BRCA1/2 status, ER status, and the like. Similarly, in a triple co-culture where PDC cells are from a patient having T-cell acute lymphoblast leukemia, (ALL), the SC cell can also be an ALL cell, e.g., a Jurkat cell line or CCRF-CEM, each of which is an immortalized cell line of human T lymphocytes used as a model of ALL. Further, in a triple co-culture where PDC cells are from a patient having acute myeloid leukemia (AML), the SC cell can be an immortalized cell line of a human myeloid cell, such as K562, KG-1, LAMA84, MEG-01, AR-230, Kasumi-6, or HL-60.

The selection of an appropriate SC cell depends on the type of cancer (e.g., tissue of origin, genetic classification, etc.) of the tumor from which the PDC cell is obtained. Thus, the SC cell can be selected on a case by case basis. A wide variety of cancer cell lines suitable for use as SC cells are available in the art. For example, SC cells can be obtained from a database of cancer cell lines described in Cancer Cell Line Encyclopedia, available at world-wide-website: portals.broadinstitute.org/ccle. Similarly, cancer cell lines suitable for use as SC cells, and classified by tissue type, can be obtained from those available from the American Type Culture Collection (ATCC), described at: world-wide-website: atcc.org/en/Landing Pages/Cancer_and_Normal_Cell Lines by_Tissue_Type.aspx.

The following table (Table 2) provides examples of cancer types of PDC cells and corresponding cancer cell lines that can be used as a SC cell.

| Patient-Derived | Cancer Cell Line Suitable for Use as | |
|---|---|---|
| Cancer Cell Type | System Control Cell | Species |
| Adrenal | NCI-H295R | Human |
| Bladder | 5637, HT-1376, J82, SW 780, T24, T24-Luc-Neo, T24P | Human |
| Brain | BT142, D54-Luc, DBTRG, LN-18, LN-229, M059K, SF-295, | Human |
| | SF-539, SF-767, SNB-19, U-251, U-87 MG | |
| Cervical | Ca Ski, HeLa, KB | Human |
| Colon | C2BBel, Caco-2, COLO 205, DLD-1, HCC2998, HCT-116, | Human |
| | HCT-15, HCT-8, HT-29, LoVo, LS 174T, LS411N, | |
| | NCI-H508, SW-480, SW-620 | |
| Erythroleukemia | HEL, HEL 92.1.7, | Human |
| Gastric | GIST-T1, NCI-N87, NUGC-4, | Human |
| Head and Neck | CAL 27, FaDu | Human |
| [squamous cell | | |
| carcinoma] | | |
| Leukemia [Acute | HL-60 | Human |
| Promyelocytic] | | |
| Leukemia [AML] | EOL-1, Kasumi-1, Kasumi-3, MOLM-13, | Human |
| | MV-4-11, NOMO-1, THP-1 | |
| Leukemia [B-ALL] | NALM6, NALM6-Luc-MCh-Puro, Reh, | Human |
| Leukemia [CML] | K-562 | Human |
| Leukemia [Plasma | ARH-77 | Human |
| Cell] | | |
| Leukemia [T-ALL] | CCRF-CEM, Jurkat, Jurkat-Clone E6-1, MOLT-4 | Human |
| Liver | Hep 3B2.1-7, Hep G2 | Human |
| Lung | NCI-H596 | Human |
| [Adenosquamous] | | |
| Lung [Anaplastic | Calu-6 | Human |
| Carcinoma] | | |
| Lung | NCI-H322M | Human |
| [Bronchioalveolar] | | |
| Lung [NSCLC] | A549, A549-Luc-C8, Calu-1, Calu-3, HCC827, NCI-H125, | Human |
| | NCI-H1299, NCI-H1650, NCI-H1703, NCI-H1975, | |
| | NCI-H2110, NCI-H2122, NCI-H23, NCI-H292, | |
| | NCI-H3122, NCI-H441, NCI-H460, | |
| | NCI-H522, PC-9 | |
| Lung [SCLC] | DMS 114, NCI-H446, NCI-H69, NCI-H82, SHP-77 | Human |
| Lung [Squamous] | EBC-1, SK-MES-1 | Human |
| Lymphoma | RL | Human |
| [B-ALL] | | |
| Lymphoma | DB, DB/M2, GRANTA-519 | Human |
| [B-Cell] | | |
| Lymphoma | Farage | Human |
| [B-NHL] | | |
| Lymphoma | B-JAB, Daudi, Daudi-Luc-mCh-Puro, NAMALWA, | Human |
| [Burkitt's] | Raji, Raji-Luc, Ramos, Ramos-Luc | |
| Lymphoma | HuT 78 | Human |
| [Cutaneous T Cell - | | |
| Sezary Syndrome] | | |
| Lymphoma | HT, SU-DHL-6 | Human |
| [Diffuse Mixed] | | |
| Lymphoma | OCI-Ly1 LN, Pfeiffer, SU-DHL-10, SU-DHL-16, | Human |
| [DLBCL] | SU-DHL-8, TMD8, WSU-DLCL2 | |
| Lymphoma | WSU-FSCCL, | Human |
| [Follicular] | | |
| Lymphoma | NK-92MI | Human |
| [Malignant NHL; | | |
| NK-Cell] | | |
| Lymphoma | KARPAS 299 | Human |
| [T-NHL] | | |
| Macrophage | J774A.1, RAW 264.7 | Mouse |
| Mammary/Breast | BT-20, BT-474, HCC1395, HCC70, Hs 578Bst, Hs 578T, MCF | Human |
| | 10A, MCF-7, MDA-MB-231, MDA-MB-361, MDA-MB-453, | |
| | MDA-MB-468, MX-1, MX-1-Luc, SK-BR-3, T47D, | |
| | UISO-BCA-1, ZR-75-1 | |
| Melanoma | A2058, A375, COLO 829, G-361, LOX-IMVI, M14, MDA-MB- | Human |
| | 435S, OCM-1, OCM-1-Luc-mCh-Puro, PA-NUT, SK-MEL-28, | |
| | SK-MEL-28-Luc-mCh-Puro, SK-MEL-5, UACC-62, | |
| | WM-115, WM-266-4 | |
| Myeloma | JJN-3-Luc, NCI-H929, | Human |
| | OPM-2, RPMI 8226, U266B1 | |
| Neuroblastoma | SK-N-AS, SK-N-FI, SK-N-SH | Human |
| Neuroendocrine | MKL-1 | Human |
| Skin | | |
| Ovarian | A2780, A2780-Luc, IGROV1, IGROV1-Luc-Mch-Puro, | Human |
| | OVCAR-4, OVCAR-5, OVCAR-5-Luc-mCh-Puro, OVCAR-8, | |
| | OVCAR-8-Luc-mCh-Puro, SK-OV-3 (Subcutaneous), | |
| | SK-OV-3-Luc-D3 (Intraperitoneal) | |
| | ID8, ID8-Luc-mCh-Puro | Mouse |
| Pancreatic | Bx-PC-3, Capan-1, Capan-2, KP4, MIA PaCa-2, | Human |
| | PANC-1, SU-86.86, SW 1990 | |
| | Pan02, Pan03 | Mouse |
| Placental | BeWo | Human |
| Choriocarcinoma | | |
| Prostate | 22Rv1, CWR-22-R, DU 145, LnCap, LnCap | Human |
| | clone FGC, PC-3, VCaP | |
| Renal | 769-P, 786-O, A-498, ACHN, Caki-1, TK-10 | Human |
| Sarcoma | A-673, HT-1080, MG-63, Saos-2, SJSA-1, SW 872 | Human |
| Thyroid | MB-1, TT | Human |
| Vulva | SK-LMS-1 | Human |

Like the TC cells, SC cells can be genetically modified to provide a detectable marker (“tag”) to facilitate analysis. Such detectable markers include a reporter protein, such as a fluorescent protein. SC cells can also optionally be genetically modified to express a selection marker, such as drug resistance marker, with the proviso that the drug resistance is to a drug different than that of the test agent(s) to be screened in the assay using the triple co-culture system. Accordingly, the discussion above with respect to genetic modification of TC cells to express a detectable marker (“tag”) and/or selection marker is also applicable to the genetic modification of SC cells.

In certain embodiments, SC cells are genetically modified to express a fluorescent protein, such as green fluorescent protein, red fluorescent protein, yellow fluorescent protein, blue fluorescent protein, or orange fluorescent protein. Examples of such fluorescent proteins include, but are not limited to: green fluorescent proteins such as, e.g., GFP (wt), Dasher, EGFP, Emerald, Superfolder GFP, Azami Green, mWasabi, TagGFP, TurboGFP, AcGFP, ZsGreen, T-Sapphire; blue fluorescent proteins such as, e.g., EBFP, EBFP2, Azurite, mTagBFP; cyan fluorescent proteins, such as, e.g., ECFP, mECFP, Cerulean, mTurquoise, CyPet, AmCyanl, Midori-Ishi Cyan, TagCFP, mTFP1 (Teal); yellow fluorescent proteins such as, e.g., EYFP, Topaz, Venus, mCitrine, YPet, TagYFP, PhiYFP, ZsYellowl, mBanana; orange fluorescent proteins, such as, e.g., Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, mTangerine; and red fluorescent proteins, such as e.g., Cayenne, mRuby, mApple, mStrawberry, AsRed2, mRFP1, JRed, mCherry, HcRedl, mRaspberry, dKeima-Tandem, HcRed-Tandem, mPlum, AQ143.

In certain embodiments, a tripe co-culture comprises a TC cell genetically modified to express a first detectable marker (e.g., reporter protein) and an SC cell genetically modified to express a second detectable marker (e.g., reporter protein), where the first and second detectable markers are different. For example, a TC cell and an SC cell can be genetically modified so that each expresses a different fluorescent protein. In such embodiments, the first and second detectable markers can be used to distinguish the TC cells from SC cells, e.g., in a flow cytometer.

SC cells can be used without long term storage, or can be stored by freezing according to methods well known in the art.

### Triple Co-Culture Compositions

As noted above, a triple-co-culture compositions disclosed herein comprises a first cell, which is a patient-derived cancer cell (PDC cell); a second cell, which is a non-tumor derived cell that supports growth and/or viability of the first cell (TC cell); and a third cell, which is a tumor-derived cell that is not derived from the patient and that exhibits sensitivity to at least one anti-cancer drug or drug combination (SC cell). Triple co-culture compositions of the present disclosure can provide a model of the microenvironment of a tumor (e.g., in a human patient), with PDC cells and TC cells as proxies for the cancerous cells (PDC cells) and non-cancerous, normal cells (TC cells) in the tumor microenvironment, and with SC cells serving as an internal positive control for a cancer cell having a known sensitivity to the test agent to be tested. In some cases, the TC cell, TC cell, and SC are each human cells (e.g., primary human cells (e.g., for PDC cells) or human cell lines (e.g., for TC cells and/or SC cells).

In the triple co-culture composition, the TC cell supports the growth of the PDC cells. Such support could be physical support and/or support provided via various secreted factors produced by the TC, such as growth factors. In addition to TC cells, the SC cells can also support the growth of the PDC cells. For example, SC cells can contribute to confluency of the cells and, therefore, providing support for viability and/or growth of PDC cells.

### Assembly of Triple Co-Cultures

Triple co-culture compositions of the present disclosure can be an adherent co-culture, a suspension culture, or a combination of adherent cells and non-adherent cells. Where the triple co-culture is a combination of adherent cells and non-adherent cells, at least one or two of the TC cell, SC cell and PDC cell is non-adherent. For example, the TC cell may be an adherent cell, while the PDC cell and the SC cell are non-adherent cells. Triple co-culture compositions can also be assembled into three-dimensional (3D) cellular aggregates, such as spheroids or tumoroids.

As used herein, “spheroid” refers to a cell aggregate that can mimic 3D cellular arrangement in a tissue. Similarly, “tumoroid” refers to a cell aggregate that can mimic 3D cellular arrangement in a tumor. Thus, in some cases, triple co-culture compositions can also be assembled into 3D tumoroids containing PDC cells, TC cells, and SC cells. In some cases of preparing such triple co-cultures in cellular aggregates, such as tumoroids, the TC cells, SC cells, and PDC cells can be added sequentially or simultaneously. In 3D tumoroids, the triple co-culture can be prepared such that the PDC cells are in excess of each of TC cells and SC cells, and/or the PDC cells are in excess of the combined total number of TC cell and SC cells. The ratio of the number of SC cells to PDC cells, the ratio of the number of TC cells to PDC cells, and the ratio of the total SC cells and TC cells to the PDC cells can be selected based on different factors, such as the cell types of each of the PDC cells, SC cells, and TC cells.

While the SC cells, TC cells, and PDC cells in 3D tumoroids are usually adherent cells, the triple co-culture composition might also contain non-adherent cells derived from the patient's tumor microenvironment. Such non-adherent cells can be infiltrating immune cells. If such non-adherent cells are present, the non-adherent cells are forced to become a part of the tumoroid by a magnetic field or by a centrifugal force in a U-bottom cell-repellent plate.

The cells in the cellular aggregates can adhere to each other via extracellular matrix (ECM) components. The ECM components can be added to the cell culture media at a low concentration, for example, between 0.1% and 5%, such as between 1% and 4%, between 1.5% and 3.5%, between 2% and 3% or about 2.5%. Non-limiting examples of ECM components that can support cellular aggregates include collagen, elastin, fibronectin, and laminin. Commercially available ECMs such as Matrigel™, MaxGel™, or Geltrex™ can also be used. The cell culture media can be additionally supplemented with fetal bovine serum or human serum.

Additional ECM components that can be used in the cellular aggregates are known in the art and use of such ECM is within the purview of this disclosure.

In some cases, the cellular aggregates in the triple co-culture system can contain patient derived non-cancer cells. Presence of such patient derived non-cancer cells mimics tumor microenvironment of the patient and can facilitate identification of cancer therapeutics that would work in the tumor microenvironment of the patient.

In some cases, such tumoroids can be assembled using cell-repellent microwell plates, for example, cell-repellent U-bottom plates. Cell-repellent plates contain cell-repellent surfaces that do not allow cell adhesion and, therefore, the cells added to the wells collect at the bottom of the wells to form cellular aggregates.

Cell-repellent plates typically contain a coating of a cell-inert substances, such as polymers that prevent the cells' attachment to the plates. Such polymers typically have extremely low surface energy and the cell surface proteins do not attach to those polymers. Non-limiting examples of such polymers are poly(tetrafluoroethylene) (PTFE), polystyrene (PS), and polyurethane (PU).

Examples of cell-repellent polymers include poly (2-hydroxyethyl methacrylate) (PHEMA) and poly (ethylene glycol) (PEG) and their derivatives. These are neutral hydrophilic polymers and contain hydrophilic hydroxyl groups or oxyethyl groups. These groups provide strong surface hydration ability and, therefore, the water molecules for a barrier due to strong hydrogen bonding between the polymer and water. This water barrier prevents cell surface proteins from adsorbing to the surface.

Another example of cell-repellent polymer includes polyelectrolytes such as polyanion and zwitterionic polyelectrolytes. Mammalian cells bear negative charge on their surfaces. Therefore, the electrostatic repulsion between the cell surface and the polymer surface prevents the attachment of the cells to the surface.

Settlement of the cells at the bottom of such wells of a cell-repellent plate can be facilitated by centrifugation of the plate. Permanent cell aggregation can be facilitated by adding ECM components.

Tumoroids can also be generated using magnetic bioprinting with or without the addition of ECM components. Magnetic bioprinting comprises embedding PDC cells, TC cells, and SC cells with biologically functionalized magnetic nanoparticles that render the cells magnetic. Once magnetic, the cells can be assembled/printed into 3D patterns of interest using external magnetic field. When used, the ECM components, can consolidate the formation, stability and viability of spheroids or aggregates.

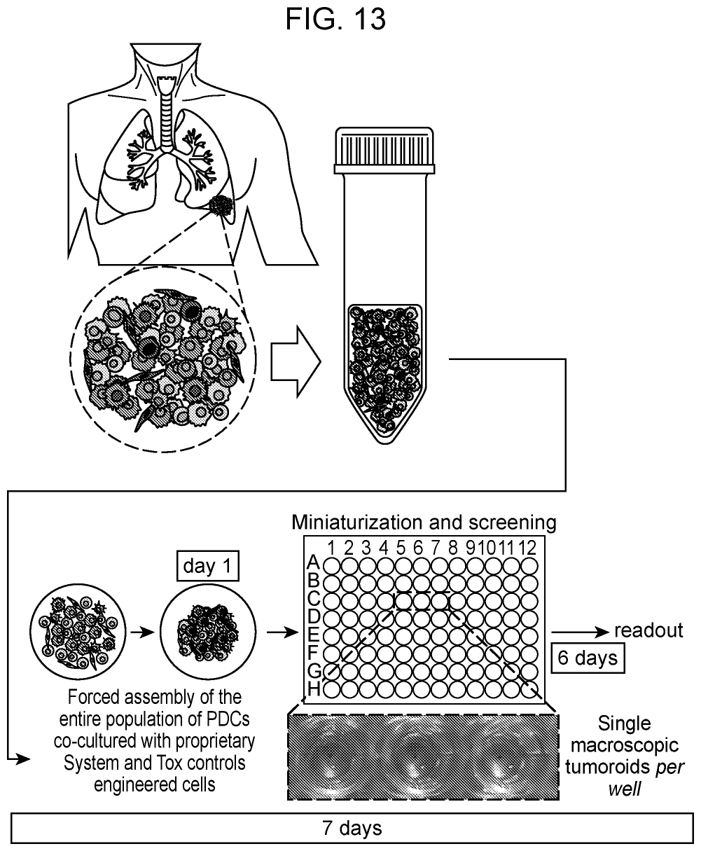

In some cases, preparations containing PDC cells can also contain other non-cancerous cells from a patient's tumor. Such non-cancerous patient cells may include healthy stromal cells, infiltrating immune cells, endothelial cells, and other patient cells present in the tumor microenvironment of the patient. Therefore, the 3D cellular arrangement produced from such preparations mimic tumor microenvironment of the patient. Such 3D cellular arrangements that mimic tumor microenvironment of a patient are referenced herein as “patient micro-avatar” (PMA). An exemplary schematic representation of producing PMAs is provided in FIG. 13.

Thus, in some cases, the disclosure provides a method for preparing a triple co-culture composition by mixing the first cell, the second cell, and the third cell and facilitating the formation of the cellular aggregate.

When magnetic printing is used, facilitating the formation of the cellular aggregate comprises incubating the first cell, the second cell, and the third cell with biologically functionalized magnetic nanoparticles that render the cells magnetic and then applying an external magnetic field to assemble the magnetic first cell, the second cell, and the third cell into the cellular aggregate.

When cell-repellant plates are used, facilitating the formation of the cellular aggregate comprises applying the first cell, the second cell, and the third cell into a container having a cell-repellent inner surface and incubating the cells for a sufficient period to allow the cells added to the container to coalesce into the cellular aggregate.

When patient derived non-cancer cells are included in the cellular aggregates, such cells are mixed with the first cell, the second cell, and the third cell.

In one embodiment, a triple co-culture composition containing adherent cells is sequentially assembled. For example, TC cells can be seeded in a culture dish and incubated for a period of time sufficient to establish an adherent cell culture, followed by addition of SC cells and PDC cells, either at around the same time or sequentially after a further period of incubation. Alternatively, SC cells can be seeded in a culture dish and incubated for a period of time sufficient to establish an adherent cell culture, followed by addition of TC cells and PDC cells either at around the same time or sequentially after a further period of incubation. In some embodiments, PDC cells are added after TC cells have been established in culture, and may be added in the last step of sequential assembly such that the established adherent TC cell culture (and, optionally, established SC cell culture) present in the culture dish provides an environment most supportive of PDC cell viability and growth.

In one example of sequential assembly of a triple co-culture containing adherent cells, TC cells is first seeded at low to medium confluency in a culture dish (e.g., well of, e.g., a multiwell plate). “Low to medium confluency” refers to seeding of TC cells at about 15 to 35 percent of full confluency. For example, a single well from a 96-mw plate having an area of 0.3 cm2 would be considered at full confluency with about 30,000 to 100,000 cells depending on the cell size. In general, seeding TC cells at low to medium confluency in a single well from a 96-mw plate constitutes plating between 5,000 and 20,000 cells. Cell culture plates having larger sizes could be seeded with appropriate number of cells accordingly and such calculations can be made by extrapolating the total culture area.

After the TC cells are established in an adherent culture, e.g., about 5 to 24 hours after seeding the TC cells, SC cells and PDC cells can be added together or sequentially. For example SC cells and PDC cells can be added to the TC cell-containing culture at the same time or within an interval of between 1 hour to 24 hours, 2 hours to 20 hours, 6 hours to 18 hours, 8 hours to 16 hours, or within about 12 hours.

Where the triple co-culture composition contains a mixture of adherent and non-adherent cells, the culture can be sequentially assembled by first seeding at least a first adherent cell type (e.g., an adherent TC cell), followed by a period of incubation sufficient to allow establishment of the adherent culture, followed by addition of the other cells (e.g., SC cell and PDC cell) either sequentially or at about the same time.

Where the TC cell, SC cell, and PDC cells are adherent cells, or in which the culture is a mixture of adherent and non-adherent cells, the triple co-culture can be prepared such that the PDC cells are in excess of each of TC cells and SC cells, and/or the PDC cells are in excess of the combined total of TC cell and SC cells. The ratio of the number of SC cells to PDC cells, ratio of the number of TC cells to PDC cells, and the ratio of total SC and TC cells to PDC cells can be selected according to a variety of different factors, such as the cell types of each of the PDC, SC, and TC cells used, and may vary as to the type of culture, e.g., whether the culture involves adherent cells of cells in suspension culture. In cases when the amount of viable cells obtained from the patient biopsy is limited, the ratio of SC/PDC cells can be altered by increasing the number of PDC cells.

For example, in adherent cell cultures, the SC cells and PDC cells provided in the co-culture may be at substantially equal numbers (e.g., at a 1:1 ratio), or the PDC cells may be in excess to the SC cells. For example, the ratio of SC cells to PDC cells can be about 1:2, about 1:3, about 1:4, about 1:5, about 1:6, about 1:7, about 1:8, about 1:9, about 1:10, about 1:11, or about 1:12, or in a range of a ratio of from about 1:2 to 1:3, about 1:2 to 1:4, about 1:2 to 1:5, about 1:2 to 1:6, about 1:2 to 1:7, about 1:2 to 1:8, about 1:2 to 1:9, about 1:2 to 1:10, about 1:2 to 1:11, or about 1:2 to 1:12.

For example, in adherent cell cultures, the TC cells and PDC cells provided in the co-culture may be at substantially equal numbers (e.g., at a 1:1 ratio), or the PDC cells may be in excess to the TC cells. For example, the ratio of TC cells to PDC cells can be about 1:2, about 1:3, about 1:4, about 1:5, about 1:6, about 1:7, about 1:8, about 1:9, about 1:10, about 1:11, or about 1:12, or in a range of a ratio of from about 1:2 to 1:3, about 1:2 to 1:4, about 1:2 to 1:5, about 1:2 to 1:6, about 1:2 to 1:7, about 1:2 to 1:8, about 1:2 to 1:9, about 1:2 to 1:10, about 1:2 to 1:11, or about 1:2 to 1:12.

For example, in adherent cell cultures, the total TC cells and SC cells in the co-culture can be substantially the same as the total PDC cells (e.g., at a 1:1 ratio), or the PDC cells may be in excess of the total of SC cells TC cells. For example, the ratio of total number of TC cells and SC cells to PDC cells can be about 1:2, about 1:3, or about 1:4, or in a range of a ratio of from about 1:1 to 1:2, about 1:1 to 1:3, or about 1:1 to 1:4.

As noted above, where the triple co-culture composition contains a mixture of adherent and non-adherent cells, the culture can be sequentially assembled by first seeding at least a first adherent cell type (e.g., an adherent TC cell), followed by a period of incubation sufficient to allow establishment of the adherent cells. Then the second type of adherent cells, if included, can be seeded, followed by a period of incubation sufficient to allow establishment of the second adherent cells and then the only non-adherent cells are added. Also, the two adherent cell types can be added at about the same time and incubated to establish the adherent cells. If only one cell type is adherent, the adherent cells are first established, followed by addition of the suspension cells either sequentially or at about the same time.

Any one or two of the SC cells, TC cells, and PDC cells can be adherent and remaining one or two types of cells can be suspension cells. For example, TC cells can be adherent cells and SC cells and PDC cells can be non-adherent cells. Also, SC cells can be adherent cells and TC cells and PDC cells can be non-adherent. Moreover, PDC cells can be adherent cells and SC cells and PDC cells can be non-adherent. Further, TC cells and SC cells can be adherent cells and PDC cells can be non-adherent cells. Furthermore, TC cells and PDC cells can be adherent cells and SC cells can be non-adherent cells. Finally, SC and PDC cells can be adherent cells and TC cells can be non-adherent.

Where the culture is a mixture of adherent and non-adherent cells, the triple co-culture can be prepared such that the PDC cells are in excess of each of TC cells and SC cells, and/or the PDC cells are in excess of the combined total of TC cell and SC cells. The ratio of the number of SC cells to PDC cells, ratio of the number of TC cells to PDC cells, and the ratio of total SC and TC cells to PDC cells can be selected according to a variety of different factors, such as the cell types of each of the PDC, SC, and TC cells used, and may vary as to the type of culture, e.g., whether the culture involves adherent cells of cells in suspension culture. In cases when the number of viable cells obtained from the patient biopsy is limited, the ratio of SC/PDC cells can be altered by increasing the number of SC cells.

When the triple co-culture comprises a mixture of adherent and non-adherent cells, the SC cells and PDC cells provided in the co-culture may be at substantially equal numbers (e.g., at a 1:1 ratio), or the PDC cells may be in excess to the SC cells. For example, the ratio of SC cells to PDC cells can be about 1:2, about 1:3, about 1:4, about 1:5, about 1:6, about 1:7, about 1:8, about 1:9, about 1:10, about 1:11, or about 1:12, or in a range of a ratio of from about 1:2 to 1:3, about 1:2 to 1:4, about 1:2 to 1:5, about 1:2 to 1:6, about 1:2 to 1:7, about 1:2 to 1:8, about 1:2 to 1:9, about 1:2 to 1:10, about 1:2 to 1:11, or about 1:2 to 1:12.

Also, when the triple co-culture comprises a mixture of adherent and non-adherent cells, the TC cells and PDC cells provided in the co-culture may be at substantially equal numbers (e.g., at a 1:1 ratio), or the PDC cells may be in excess to the TC cells. For example, the ratio of TC cells to PDC cells can be about 1:2, about 1:3, about 1:4, about 1:5, about 1:6, about 1:7, about 1:8, about 1:9, about 1:10, about 1:11, or about 1:12, or in a range of a ratio of from about 1:2 to 1:3, about 1:2 to 1:4, about 1:2 to 1:5, about 1:2 to 1:6, about 1:2 to 1:7, about 1:2 to 1:8, about 1:2 to 1:9, about 1:2 to 1:10, about 1:2 to 1:11, or about 1:2 to 1:12.

Similarly, when the triple co-culture comprises a mixture of adherent and non-adherent cells, the total TC cells and SC cells in the co-culture can be substantially the same as the total PDC cells (e.g., at a 1:1 ratio), or the PDC cells may be in excess of the total of SC cells TC cells. For example, the ratio of total number of TC cells and SC cells to PDC cells can be about 1:2, about 1:3, or about 1:4, or in a range of a ratio of from about 1:1 to 1:2, about 1:1 to 1:3, or about 1:1 to 1:4.

In some cases, the triple co-culture composition is a suspension co-culture. In such suspension co-cultures, the TC cells, SC cells, and PDC cells can be added sequentially as described above, or may be added at the same time. In one example, the TC cells are added to the suspension culture prior to adding PDC cells. For example, the TC cells are added to the suspension culture and within an interval of between 1 hour to 24 hours, 2 hours to 20 hours, 6 hours to 18 hours, 8 hours to 16 hours, or within about 12 hours, the SC cells or both SC cells and PDC cells are added to the suspension culture. In one embodiment, the TC cells can be cultured in suspension for a period of time sufficient to provide a TC cell-conditioned culture medium prior to adding the PDC cells.

In suspension cultures, the triple co-culture can be prepared such that the PDC cells are in excess of each of TC cells and SC cells, and/or the PDC cells are in excess of the combined total of TC cell and SC cells. The ratio of the number of SC cells to PDC cells, ratio of the number of TC cells to PDC cells, and the ratio of total SC and TC cells to PDC cells can be selected according to a variety of different factors, such as the cell types of each of the PDC, SC, and TC cells used.

For example, in suspension cell cultures, the SC cells and PDC cells provided in the co-culture may be at substantially equal numbers (e.g., at a 1:1 ratio), or the PDC cells may be in excess to the SC cells. For example, the ratio of SC cells to PDC cells can be about 1:2, about 1:3, about 1:4, about 1:5, about 1:6, about 1:7, about 1:8, about 1:9, about 1:10, about 1:11, or about 1:12, or in a range of a ratio of from about 1:2 to 1:3, about 1:2 to 1:4, about 1:2 to 1:5, about 1:2 to 1:6, about 1:2 to 1:7, about 1:2 to 1:8, about 1:2 to 1:9, about 1:2 to 1:10, about 1:2 to 1:11, or about 1:2 to 1:12.

For example, in suspension cell cultures, the TC cells and PDC cells provided in the co-culture may be at substantially equal numbers (e.g., at a 1:1 ratio), or the PDC cells may be in excess to the TC cells. For example, the ratio of TC cells to PDC cells can be about 1:2, about 1:3, about 1:4, about 1:5, about 1:6, about 1:7, about 1:8, about 1:9, about 1:10, about 1:11, or about 1:12, or in a range of a ratio of from about 1:2 to 1:3, about 1:2 to 1:4, about 1:2 to 1:5, about 1:2 to 1:6, about 1:2 to 1:7, about 1:2 to 1:8, about 1:2 to 1:9, about 1:2 to 1:10, about 1:2 to 1:11, or about 1:2 to 1:12.

For example, in suspension cell cultures, the total TC cells and SC cells in the co-culture can be substantially the same as the total PDC cells (e.g., at a 1:1 ratio), or the PDC cells may be in excess of the total of SC cells TC cells. For example, the ratio of total number of TC cells and SC cells to PDC cells can be about 1:2, about 1:3, or about 1:4, or in a range of a ratio of from about 1:1 to 1:2, about 1:1 to 1:3, or about 1:1 to 1:4.

In 3D tumoroids, the SC cells and PDC cells provided in the triple co-culture may be in a substantially equal numbers (e.g., at a 1:1 ratio), or the PDC cells may be in excess to the SC cells. For example, the ratio of SC cells to PDC cells can be about 1:2, about 1:3, about 1:4, about 1:5, about 1:6, about 1:7, about 1:8, about 1:9, about 1:10, about 1:11, or about 1:12, or in a range of a ratio of from about 1:2 to 1:3, about 1:2 to 1:4, about 1:2 to 1:5, about 1:2 to 1:6, about 1:2 to 1:7, about 1:2 to 1:8, about 1:2 to 1:9, about 1:2 to 1:10, about 1:2 to 1:11, or about 1:2 to 1:12.

### Cell Culture Media

A triple co-culture composition can include standard culture medium components, such as amino acids, vitamins, inorganic salts, a carbon energy source, and a buffer. Other standard cell culture components that may be included in the culture include hormones, such as progesterone, proteins, such as albumin, catalase, insulin, and transferrin.

Cell culture media for a triple co-culture of the present disclosure can be selected from any of a variety available in the art, and can be selected according to, for example, the types of TC cells, SC cells, and PDC cells present in a triple co-culture.

Suitable cell culture media are available commercially, and include, but are not limited to, Dulbecco's Modified Eagle Media (DMEM), Minimal Essential Medium (MEM), Knockout-DMEM (KO-DMEM), Glasgow Minimal Essential Medium (G-MEM), Basal Medium Eagle (BME), DMEM/Ham's F12, Advanced DMEM/Ham's F12, Iscove's Modified Dulbecco's Media and Minimal Essential Media (MEM), Ham's F-10, Ham's F-12, Medium 199, and RPMI 1640 Media.

In some cases, the triple co-culture composition contains human serum. The human serum can be autologous, i.e., the serum is derived from the cancer patient from whom the PDC cells in the culture were obtained. Alternatively, the triple co-culture composition contains serum derived from other sources, such as non-autologous human serum, fetal bovine serum, calf, or horse serum.

### Methods of Use of Triple Co-Cultures

The triple co-culture compositions disclosed herein can be used in a variety of methods. In some cases, such methods can be used to provide patient-specific reproducible insights of predicted patient response to cancer treatments in a high-throughput manner. Particularly, the miniaturized replicas of the patient disease (PMAs: Patient Micro-Avatars) described herein can be used to detect a response of the PDC cells to different test agents. The methods disclosed herein can also be used to identify from a set of therapeutic agents, a particular therapeutic agent, or a specific concentration of a therapeutic agent that exhibits cytotoxic effects on the PDC cells, thus providing guidance for selection of a therapy for the patient from whom the PDC cells were obtained. Examples of methods of use of the triple co-culture compositions, including the triple co-culture compositions containing PMAs as described in the present disclosure include:

A) facilitating selection of an anti-cancer therapeutic for a patient on an individualized basis (sometimes referred to as “personalized medicine”);

B) determining efficacy of a candidate anti-cancer therapeutic in a pre-clinical setting;

C) determining lead anti-cancer therapeutics from a set of test agents that are candidate anti-cancer therapeutics.

In one embodiment, the triple co-culture system of the present disclosure is used in a Drug Activity and Resistance Test (DART) to identify intrinsic vulnerabilities and resistance of PDC cells to drugs and drugs combinations.

The final design of the test varies depending on the type of sample and purpose of the screening. Several applications of DART are within pre-clinical settings. These applications comprise drug discovery project with compound libraries, candidate validation, and prioritization with small selection of candidates in drug development project, and also the selection of the most suitable patients for clinical trials.

Depending on the study, the selection of drugs and dosage can vary greatly. For example, in a screening that aims to select the best drug candidates within a small set of compounds, DART might be employed in a small subset of patients in broad-dose-response schemes at multiple time points, and, optionally, in combination with approved drugs (e.g., FDA approved drugs) currently used in the clinic for those types of patient from whom the PDC cells were obtained.

In another example, DART is used for the selection of patients for a clinical trial. In this case, DART can be implemented to efficiently screen a large number of patients, thereby increasing the likelihood of identifying patients most likely to benefit from inclusion in an early-stage clinical trial.

DART can also be used for ex-vivo testing of approved drugs for therapy selection in a clinical setting. The goal is to aid oncologists' efforts to achieve optimal therapy selection. DART results can be combined with other information, such as the patient's medical history, pathology studies, and/or with other precision oncology studies such as genomic profiling of driver mutations. In this context, available prescription drugs and drug combinations are tested for the patient. As such, the final drug matrix can vary depending on the country where DART is applied and/or on particular drugs and/or combinations that might be requested by oncologists.

### Test Agents and Selection of Doses

As noted above, “test agent” refers to an anti-cancer drug or combination of anti-cancer drugs (or candidate anti-cancer drug or combination of anti-cancer drugs) amenable for screening in an assay described herein. Candidate drugs include agents for which anti-cancer activity against a selected cancer type may not be known. “Drugs” include, for example, small molecule drugs, antibodies, nucleic acids (e.g., siRNAs, etc.), gene targeting systems (e.g., TALENs, ZFNs, CRISPR gene editing systems), and the like.

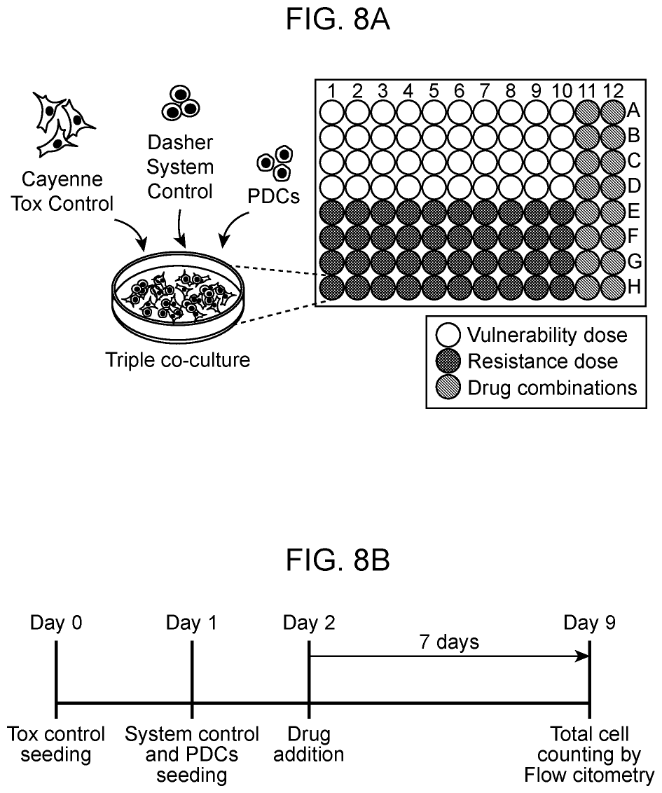

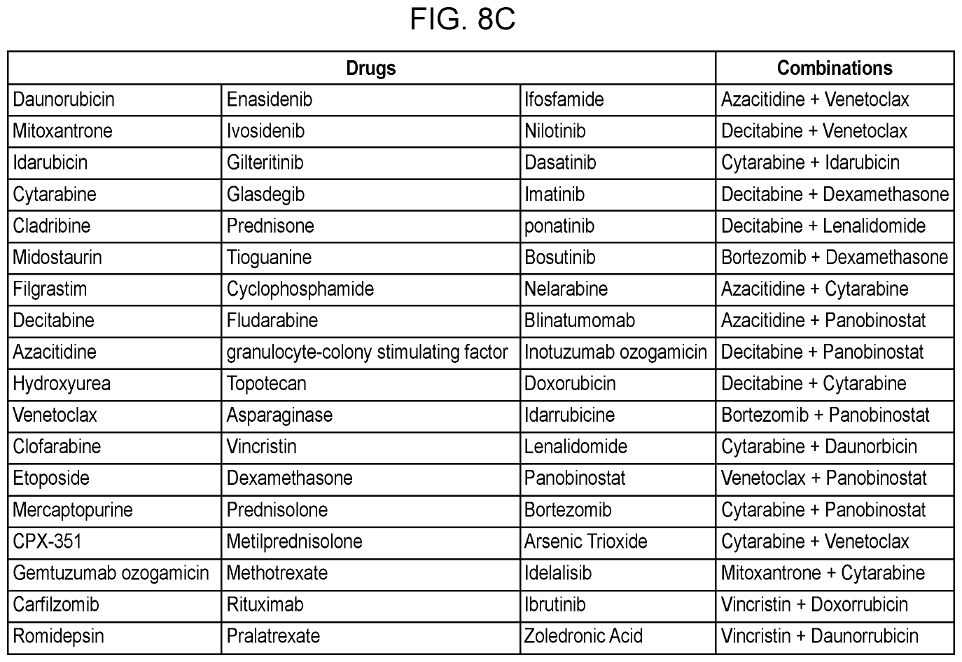

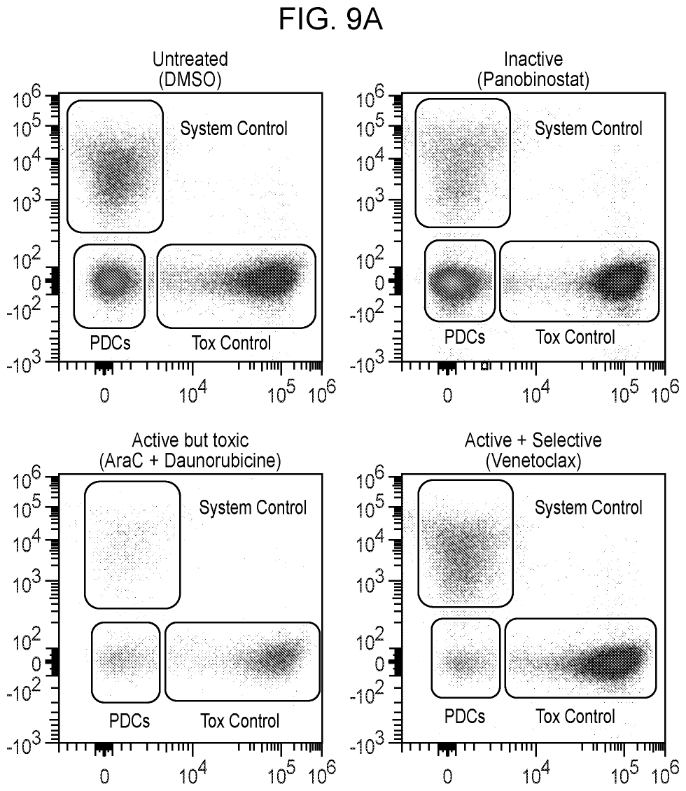

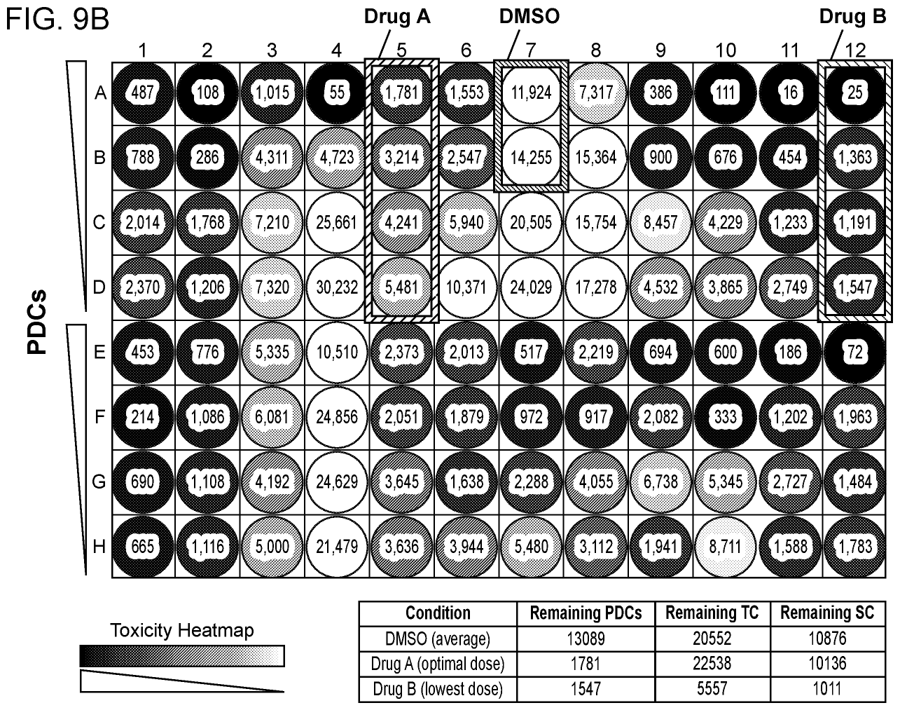

Test agents can also be selected from anti-cancer therapeutics approved for use in the clinic (e.g., FDA approved therapeutics). For example, where the PDC cells are from a patient having ALL or AML, an example of a general matrix of drugs and drug combinations suitable for use as test agents are provided in the table of FIG. 8C.

For example, as in the DART assay, the dosage strategy for approved drugs (e.g., FDA approved drugs) can involve the test of two reference doses for each drug and one optimal combination for each set of drugs (see, e.g., FIG. 8A). From these analyses, a low and high dose can be defined for each drug in the matrix.

The low dose can be selected to uncover clear vulnerabilities in patients (vulnerability proxy or “vulnerability dose”), with the understanding that only extremely sensitive patients show ex vivo sensitivity at very low doses. While this vulnerability dose strategy might result in some false negatives (patients for which their PDC cells show no sensitivity to the vulnerability dose, but which would be sensitive to the drug in vivo), the confidence on the positives should be high. Appropriate doses to be tested can be determined on a case by case basis based on the specific drug to be tested, and the types of PDC cells, TC cells, and SC cells used as well as other culture conditions.

The goal of the high doses is to identify those patients that are highly likely to be resistant to that drug in vivo (resistance proxy or “resistance dose”). While this strategy might lead to false positives (patients for which their PDC cells show ex vivo resistance but that would be sensitive to the drug in vivo), such feature will be associated with a very low rate of false negatives. Such combination of positive and negative predictive power provides valuable insights in clinical settings for oncology therapy selection, both at initial and subsequent lines of treatment. Appropriate doses to be tested can be determined on a case by case basis based on the specific drug to be tested, and the types of PDC cells, TC cells, and SC cells used as well as other culture conditions.

Doses can be based on a combination of patient database analysis from published works (see, e.g., Frismantas, et al. (2017) e-Blood “Ex vivo drug response profiling detects recurrent sensitivity patterns in drug-resistant acute lymphoblastic leukemia.” 129:26-37; and Spinner, et al. (2020) “Ex vivo drug screening defines novel drug sensitivity patterns for informing personalized therapy in myeloid neoplasms.” Blood Adv. 4:2768-2778) in combination with experimental data with wide dose-response schemes with multiple cell lines and PDC cells.

### Addition of Test Agent(s) to Triple Co-Culture

Once the triple co-culture is assembled as described above test agents are added to the culture into the culture medium. The test agents can be provided in a defined volume of a suitable medium (e.g., culture medium/DMSO), with the same medium (e.g., culture medium/DMSO without test agent) serving as a negative control. The triple co-culture is then incubated for a period of time of interest, e.g., a time sufficient to observe an effect of the test agent on the SC cell.

In general, the method involves culturing the triple co-culture composition for a first time period, then, at the conclusion of the first time period, adding, test agent to the triple co-culture and incubating the culture in the presence of the test agent for a second time period. At the conclusion of the second time period, the effect of the test agent is analyzed detecting the response of the PDC cells, TC cells, and SC cells.

In certain such cases, the first time period of culturing prior to addition of test agent is from about 12 hours to 48 hours, from about 18 hours to 48 hours days, from about 24 hours to 36 hours, or about 30 hours. In some cases, test agent is added to the culture within about 1 hour to 24 hours, about 2 hours to 20 hours, about 6 hours to 18 hours, about 8 hours to 16 hours, or within about 12 hours after adding the PDC cells to the culture.

In some cases, the second period, i.e., the period for incubating the test agent with the triple co-culture, is at least 2 days, at least 3 days, at least 5 days, at least 7 days, at least 8 days, at least 9 days, or at least 10 days, and may be from 2 days to 3 days, from 2 days to 4 days, from 2 days to 5 days, from 2 days to 6 days, from 2 days to 7 days, from 2 days to 8 day, from 5 days to 9 days, or longer. In some cases, the second period is for at least one cell cycle of the PDC cell. In some embodiments, samples are analyzed after different periods of incubation of the triple co-culture in the presence of the test agent.

Where the triple co-cultures are performed in multiwell plates (e.g., 96 or 384 multiwell plates), evaporation can result in reduction of the fluid in the wells during long-term screening experiments (e.g., over 3 days, 5 days, 7 days, or longer). Such evaporation can be counteracted by adding fluid, e.g., PBS, in the interstitial space between the wells of the plates.

### Methods of Detecting and Evaluating PDC Cell Responses in Triple Co-Cultures

After the end of the incubation with the test agent (e.g., anti-cancer drug or combination of anti-cancer drugs), the effect of exposure to the test agent on the TC cells, SC cells, and PDC cells is analyzed. Detecting the response of the TC cells, SC cells, and PDC cells to the test agent comprises detecting a response on these cells in the presence of the test agent compared to a control, i.e., in the absence of the test agent. Responses assessed can include changes in cell viability (e.g., cell death, e.g., apoptosis), cell growth (e.g., cell growth inhibition, e.g., inhibition of cell division), cell number, cell size, and/or cell morphology.

If the triple co-culture composition is an adherent culture (or a mixture of adherent and non-adherent cells), the cells are released from the support via enzymatic or mechanical action to prepare a suspension of cells in an appropriate buffer. If the triple co-culture composition is a suspension culture, the cells can be separated from the culture medium and resuspended in an appropriate buffer. Such cell suspension can be analyzed in a flow cytometer.

In one embodiment, the response of the TC cells, SC cells, and PDC cells is assessed using flow cytometry. Response of the TC cells and of the SC cells to the test agent can be assessed using a fluorescently labeled antibody that specifically binds a cell surface marker that is specific for the TC cell or for the SC cell (e.g., a native or recombinantly expressed cell surface protein), which distinguishes the TC cells from SC cells, as well as from PDC cells. Binding of the TC cell surface marker by the fluorescently labeled antibody distinguishes TC cells from SC cells and from PDC cells, and binding of the SC cell surface marker by the fluorescently labeled antibody distinguishes SC cells from TC cells and from PDC cells.

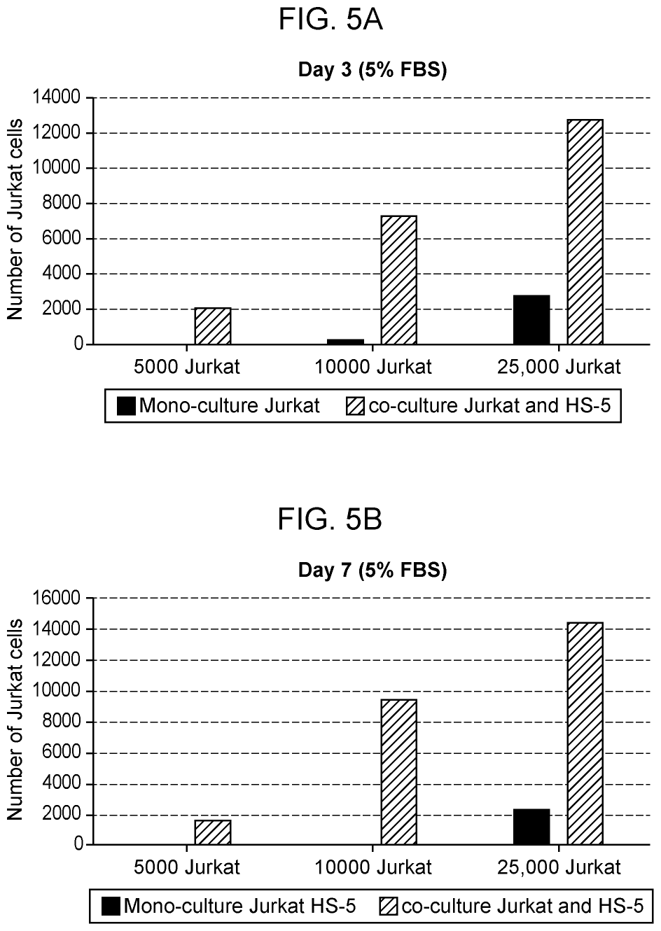

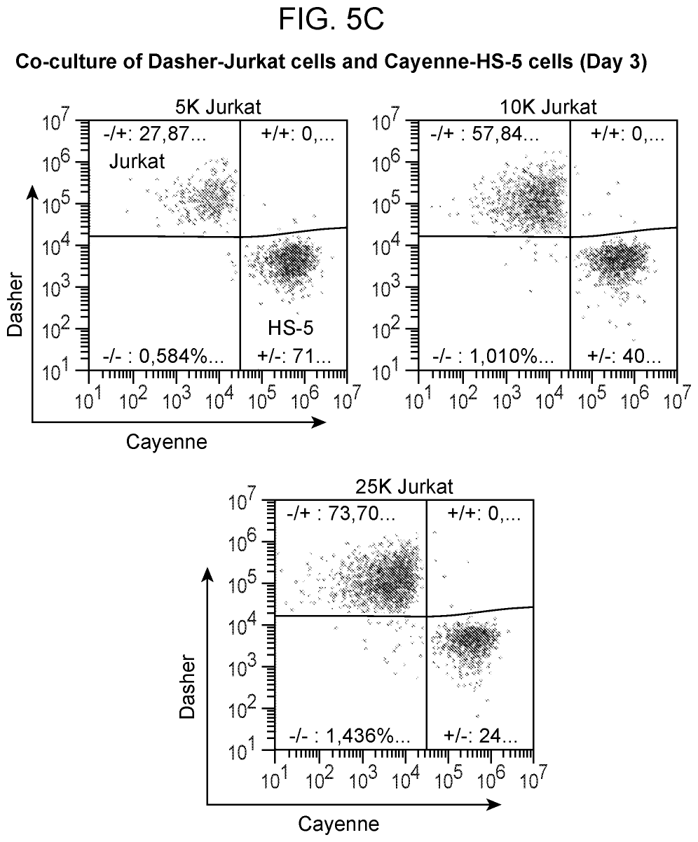

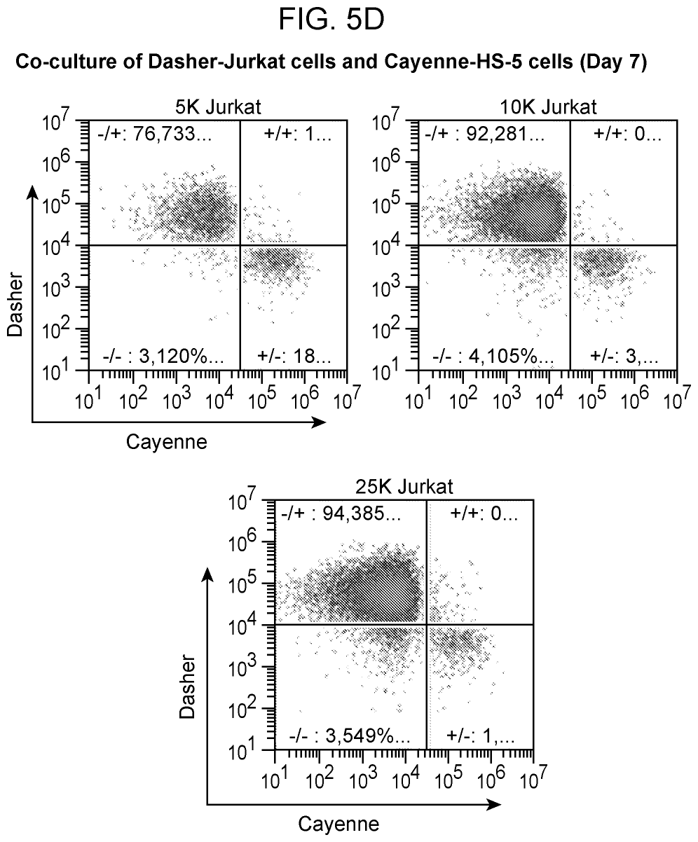

In some embodiments one or both of the TC cells and the SC cells are genetically modified to express a reporter protein, such as a fluorescent protein. Where both the TC cells and SC cells are genetically modified to express a reporter protein, such as a fluorescent protein, the reporter proteins are different between the TC cells and SC cells. For example, were the TC cells and SC cells express a fluorescent protein, the excitation and/or emission wavelengths of the fluorescent proteins are selected so that they can be distinguished by FACS. Thus, TC cells can be detected in a flow cytometer using specific fluorescence emitted by the TC cells and distinguished from SC cells, and vice versa (FIGS. 5C-5D).

If a test agent does not exhibit any adverse effects on the TC cells, the response of the TC cells in a triple co-culture in the presence of the test agent would be similar to the response of the TC cells in the absence of the test agent. On the other hand, if a test agent exhibits any adverse effects on the TC cells, the adverse response of the TC cells in a triple co-culture in the presence of the test agent would be higher than the adverse response of the TC cells in the absence of the test agent.

If a test agent does not exhibit any adverse effects on the SC cells, the response of the SC cells in a triple co-culture in the presence of the test agent would be similar to the response of the SC cells in the absence of the test agent. On the other hand, if a test agent exhibits any adverse effects on the SC cells, the adverse response of the SC cells in a triple co-culture in the presence of the test agent would be higher than the adverse response of the SC cells in the absence of the test agent.

Also, to test proper operation of the method, a triple co-culture composition is contacted with an anti-cancer test agent to which the SC cells are known to be sensitive. Therefore, in such control triple co-cultures, the adverse response of the SC cells in a triple co-culture in the presence of the anti-cancer therapeutic would be higher than the adverse response of the SC cells in the absence of the anti-cancer therapeutic and such results would indicate proper operation of the screening method. Therefore, SC cell can perform as internal controls.

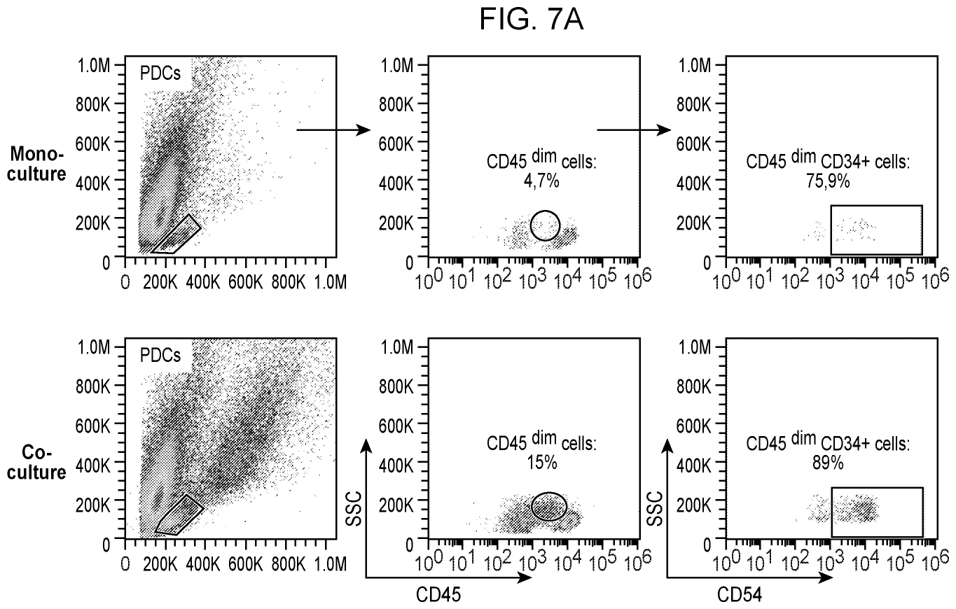

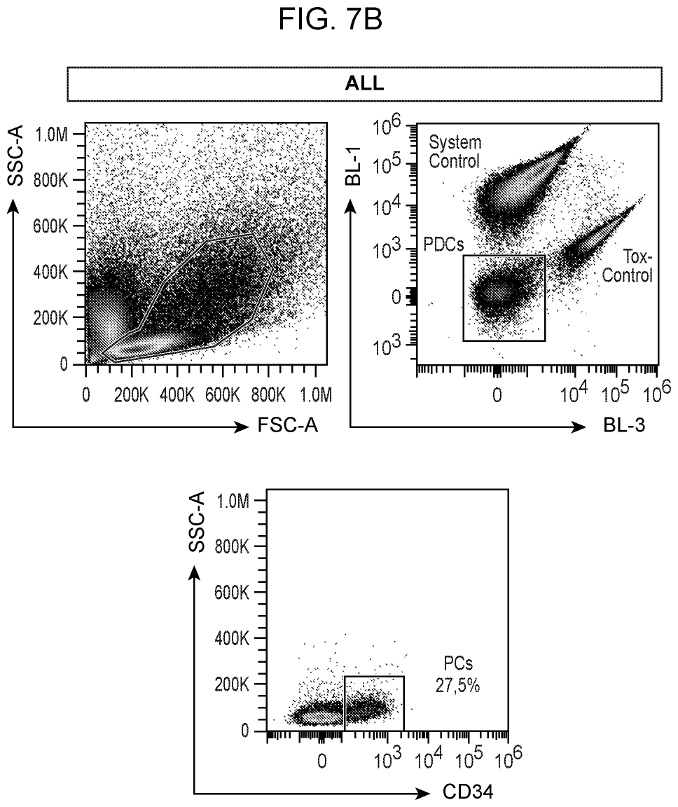

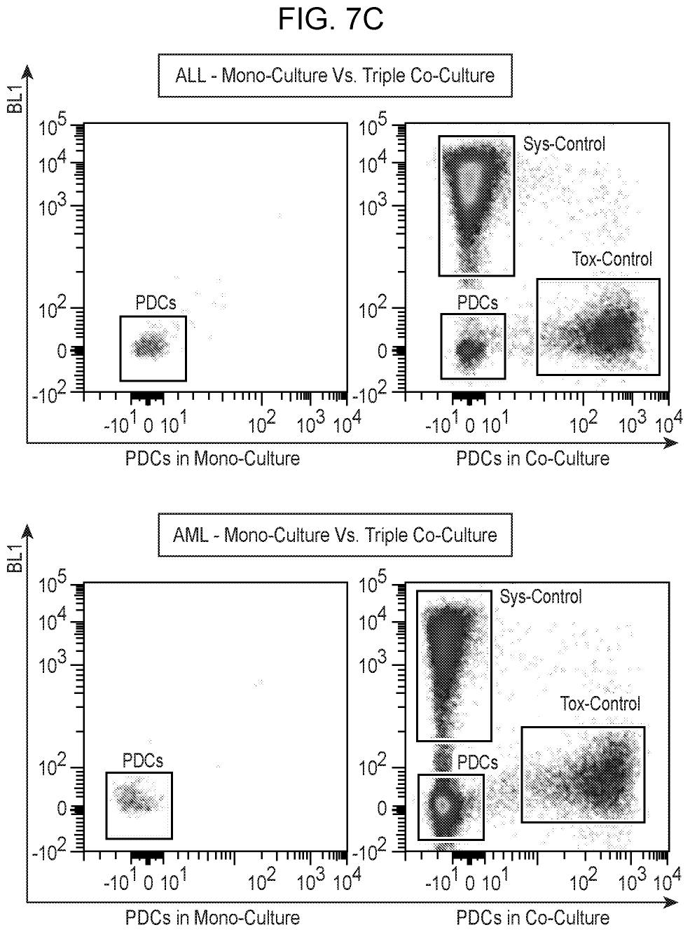

Further, the response of the PDC cells to the test agent can be distinguished from that of the TC cells and SC cells by either using a fluorescently labelled antibody that binds a PDC cell-specific cell surface marker, or by exploring the absence of a reporter protein (e.g., no fluorescent protein) in the PDC cells. For example, in the flow cytometer, the PDC cells are those lacking a fluorescence associated with the reporter proteins of the TC cells or SC cells (FIG. 7B).

For example, where a test agent does not exhibit any cytotoxic effects on the PDC cells, the response of the PDC cells in a triple co-culture in the presence of the test agent would be similar to the response of the PDC cells in the absence of the test agent. On the other hand, if a test agent exhibits any cytotoxic effects on the PDC cells, the adverse response of the PDC cells in a triple co-culture in the presence of the test agent would be higher than the adverse response of the PDC cells in the absence of the test agent.

In some cases, the response to the test agent of the PDC cells is compared to the response to the test agent of the TC cells, and the response of the SC cells as a control is considered.

For example, a test agent may induce cytotoxic effects on the PDC cells while inducing no, or acceptably lower cytotoxic, effects on the TC cells. Such results would indicate that the test agent would be suitable for treating the patient from whom the PDC cells were obtained, where such test agent may have lower or no side effects. In this case, where the test agent also induces cytotoxic effects on the SC cells, the SC cells provide confidence that the assay is functioning properly.

In another example, a test agent may induce cytotoxic effects on the PDC cells, while also inducing significant cytotoxic effects on the TC cells. Such results would indicate that the test agent may be less suitable for treating the patient, since the observations with TC cells suggest normal non-cancerous cells would be adversely affected, and the patient may experience significant side effects. If the observed cytotoxic effects on the PDC cells are low or minimal, the impact on the normal non-cancerous cells may indicate the test agent would likely provide no overall clinical benefit to the patient.

In addition to comparing the response to the test agent of the PDC cells to the response to the test agent of the TC cells, the response to the SC cells can also be considered. As noted above, in the presence of an anti-cancer therapeutic known to be effective against the SC cells, higher adverse response of the SC cells would be observed in the presence of the anti-cancer therapeutic compared to the adverse response of the SC cells in the absence of the anti-cancer therapeutic. Such higher adverse response in the presence of the anti-cancer therapeutic would indicate proper operation of the screening methods. However, if such expected cytotoxicity of the anti-cancer therapeutic is not observed on the SC cells, the results of the screening methods may need to be further verified.

### Screening a Set of Therapeutics or Candidate Therapeutics

The triple co-culture compositions and methods of detecting a response to a test agent of TC cells, SC cells, and PDC cells could be used to screen a set of therapeutics, for example, a library of compounds. Such screening can be performed to identify from a number of potential anti-cancer therapeutics the anti-cancer treatment suitable for a patient. Such screening can also be performed to identify from a library of compounds a lead compound suitable as an anti-cancer therapeutic.

Accordingly, the present disclosure provides a method for detecting tumor cell response to a set of test agents (e.g., drugs or combination of drugs) by assembling a set of triple co-cultures as disclosed herein, and where each triple co-culture in the set comprises the same PDC cells, the same TC cells, and the same SC cells. A different test agent from the set of test agents (and/or different concentrations of the same test agent from the set of test agents) is administered to each triple co-culture of the set of triple co-cultures. Following incubation for a sufficient time, the response of the PDC cells, TC cells, and SC cells in each triple co-culture is assessed, e.g., as described above.

In the context of the methods disclosed herein for detecting tumor cell response to a set of test agents, a test agent can be a specific drug in a set of drugs, or a specific concentration of a drug in a set of different concentrations of the drug. Thus, the methods disclosed herein can be used to identify, for example, one or more effective therapeutics from a set of therapeutics or one or more effective concentrations of a therapeutic.

For example, a suitable therapeutic from the set of therapeutics would induce adverse response in the PDC cells (e.g., cell death), would induce none or minimal adverse response in the TC cells, and where the SC cells is sensitive to the therapeutic tested at the concentration tested, would induce an adverse response in the SC cells (e.g., cell death).

Notwithstanding the appended claims, the present disclosure is also defined by the following embodiments:

Embodiment 1. A triple co-culture composition comprising: a first cell, wherein the first cell is a patient-derived cancer cell;
a second cell, wherein the second cell is a non-tumor derived cell that supports growth and/or viability of the first cell; and
a third cell, wherein the third cell is a tumor-derived cell that is not derived from the patient and that exhibits sensitivity to at least one anti-cancer drug or drug combination.

Embodiment 2. The triple co-culture composition of embodiment 1, wherein the first cell and the third cell are derived from the same type of cancer.

Embodiment 3. The triple co-culture composition of embodiment 1 or embodiment 2, wherein the second cell and the third cell are non-isogenic.

Embodiment 4. The triple co-culture composition of any one of embodiments 1-3, wherein the first cell, second cell, and the third cell are human cells.

Embodiment 5. The triple co-culture composition of any one of embodiments 1-4, wherein the second cell is of a non-tumoral cell type that is present in a tumor microenvironment of the first cell.

Embodiment 6. The triple co-culture composition of embodiment 5, wherein the second cell is a bone marrow stromal cell and the first cell is a patient-derived cancer cell of hematological origin.

Embodiment 7. The triple co-culture of embodiment 5, wherein the second cell is a fibroblast and the first cell is a patient-derived cancer cell from a solid tumor.

Embodiment 8. The triple co-culture composition of any one of embodiments 1-7, wherein at least one of the second cell and the third cell comprises a heterologous gene.

Embodiment 9. The triple co-culture composition of embodiment 8, wherein the second cell is labeled with a first reporter.

Embodiment 10. The triple co-culture composition embodiment 8 or embodiment 9, wherein the third cell is labeled with a second reporter.

Embodiment 11. The triple co-culture composition of embodiment 10, wherein the first reporter and the second reporter are different from each other.

Embodiment 12. The triple co-culture composition of any one of embodiments 9-11, wherein at least one of the first reporter and the second reporter is a fluorescent protein.

Embodiment 13. The triple co-culture composition of any one of embodiments 1-12, wherein the second cell and third cell promote growth of the first cell in the triple co-culture.

Embodiment 14. The triple co-culture composition of any one of embodiments 1-6 and 8-13, wherein the first cell is a blood cancer cell.

Embodiment 15. The triple co-culture composition of embodiment 14, wherein the blood cancer is a leukemia.

Embodiment 16. The triple co-culture composition of any one of embodiments 1-5 and 7-13, wherein the first cell is a cell derived from a solid tumor.

Embodiment 17. The triple co-culture composition of any one of embodiments 1 to 16, wherein the first cell, second cell, and third cell are in a cellular aggregate.

Embodiment 18. The triple co-culture composition of embodiment 17, wherein the first cell, second cell, and third cell in the cellular aggregate adhere to each other via extracellular matrix proteins.

Embodiment 19. The triple co-culture composition of embodiment 17 or embodiment 18, wherein the composition further comprises a patient derived non-cancer cell from the patient's cancer microenvironment.

Embodiment 20. The triple co-culture composition of embodiment 19, wherein the patient derived non-cancer cell from the patient's cancer microenvironment is a healthy stromal cell, an infiltrating immune cell, or an endothelial cell.

Embodiment 21. The triple co-culture composition of any one of embodiments 1-20, wherein:
i) when the first cell is a patient derived cancer cell of a certain cancer of the hematological origin; the second cell is a bone marrow stromal cell; and the third cell is a cancer cell of the certain cancer of the hematological origin; or
ii) when the first cell is a patient derived cell of a solid tumor, the second cell is a fibroblast, vascular endothelial cell, pericyte, or adipocyte, and the third cell is a cancer cell of the solid tumor.

Embodiment 22. The triple co-culture composition of embodiment 21, wherein:
i) when the first cell is a patient derived cancer cell of a certain cancer of the hematological origin selected from leukemia, lymphoma, and myeloma;
the second cell is a bone marrow stromal cell; and
the third cell is a cancer cell of the certain cancer of the hematological origin.

Embodiment 23. The triple co-culture composition of embodiment 22, wherein:
i) when the first cell is a patient derived leukemia cancer cell; the second cell is a bone marrow stromal cell; and the third cell is leukemia cancer cell;
ii) when the first cell is a patient derived lymphoma cell; the second cell is a bone marrow stromal cell; and the third cell is a lymphoma cell; or
iii) when the first cell is a patient derived myeloma cell; the second cell is a bone marrow stromal cell; and the third cell is a myeloma cell.

Embodiment 24. The triple co-culture composition of embodiment 21, wherein:
the first cell is a patient derived cancer cell of a certain solid tumor selected from acral lentiginous melanoma, adenocarcinoma, adenoma, anaplastic thyroid cancer, brain tumor, breast cancer, colon cancer, colorectal cancer, cutaneous T-cell lymphoma, epithelioid sarcoma, esophageal cancer, follicular lymphoma, gastrointestinal cancer, head and neck cancer, hepatocellular carcinoma, intraocular melanoma, melanoma, nodular melanoma, nonmelanoma skin cancer, non-small cell lung cancer, ovarian cancer, ovarian epithelial cancer, pancreatic cancer, and uveal melanoma;
the second cell is a fibroblast, vascular endothelial cell, pericyte, or adipocyte; and
the third cell is a cancer cell of the certain solid tumor.

Embodiment 25. The triple co-culture composition of embodiment 21, wherein:
i) when the first cell is a patient derived acral lentiginous melanoma cell; the second cell is a fibroblast, vascular endothelial cell, pericyte, or adipocyte; and the third cell is an acral lentiginous melanoma cell;
ii) when the first cell is a patient derived adenocarcinoma cell; the second cell is a fibroblast, vascular endothelial cell, pericyte, or adipocyte; and the third cell is an adenocarcinoma cell;
iii) when the first cell is a patient derived adenoma cell; the second cell is a fibroblast, vascular endothelial cell, pericyte, or adipocyte; and the third cell is an adenoma cell;
iv) when the first cell is a patient derived thyroid cancer cell; the second cell is a fibroblast, vascular endothelial cell, pericyte, or adipocyte; and the third cell is a thyroid cancer cell;
v) when the first cell is a patient derived brain cancer cell; the second cell is a fibroblast, vascular endothelial cell, pericyte, or adipocyte; and the third cell is a brain cancer cell;
vi) when the first cell is a patient derived breast cancer cell; the second cell is a fibroblast, vascular endothelial cell, pericyte, or adipocyte; and the third cell is a breast cancer cell;
vii) when the first cell is a patient derived colon cancer cell; the second cell is a fibroblast, vascular endothelial cell, pericyte, or adipocyte; and the third cell is a colon cancer cell;
viii) when the first cell is a patient derived colorectal cancer cell; the second cell is a fibroblast, vascular endothelial cell, pericyte, or adipocyte; and the third cell is a colorectal cancer cell;
ix) when the first cell is a patient derived cutaneous T-cell lymphoma cell; the second cell is a fibroblast, vascular endothelial cell, pericyte, or adipocyte; and the third cell is a cutaneous T-cell lymphoma cell;
x) when the first cell is a patient derived epithelial sarcoma cell; the second cell is a fibroblast, vascular endothelial cell, pericyte, or adipocyte; and the third cell is an epithelial sarcoma cell;
xi) when the first cell is a patient derived esophageal cancer cell; the second cell is a fibroblast, vascular endothelial cell, pericyte, or adipocyte; and the third cell is an esophageal cancer cell;
xii) when the first cell is a patient derived follicular lymphoma cell; the second cell is a fibroblast, vascular endothelial cell, pericyte, or adipocyte; and the third cell is a follicular lymphoma cell;
xiii) when the first cell is a patient derived gastrointestinal cancer cell; the second cell is a fibroblast, vascular endothelial cell, pericyte, or adipocyte; and the third cell is a gastrointestinal cancer cell;
xiv) when the first cell is a patient derived head and neck cancer cell; the second cell is a fibroblast, vascular endothelial cell, pericyte, or adipocyte; and the third cell is a head and neck cancer cell;
xv) when the first cell is a patient derived hepatocellular carcinoma cell; the second cell is a fibroblast, vascular endothelial cell, pericyte, or adipocyte; and the third cell is a hepatocellular carcinoma cell;
xvi) when the first cell is a patient derived intraocular melanoma cell; the second cell is a fibroblast, vascular endothelial cell, pericyte, or adipocyte; and the third cell is an intraocular melanoma cell;
xvii) when the first cell is a patient derived melanoma cell; the second cell is a fibroblast, vascular endothelial cell, pericyte, or adipocyte; and the third cell is a melanoma cell; xviii) when the first cell is a patient derived nodular melanoma cell; the second cell is a fibroblast, vascular endothelial cell, pericyte, or adipocyte; and the third cell is a nodular melanoma cell;
xix) when the first cell is a patient derived nonmelanoma skin cancer cell; the second cell is a fibroblast, vascular endothelial cell, pericyte, or adipocyte; and the third cell is a nonmelanoma skin cancer cell;
xx) when the first cell is a patient derived non-small cell lung cancer cell; the second cell is a fibroblast, vascular endothelial cell, pericyte, or adipocyte; and the third cell is a non-small cell lung cancer cell;
xxi) when the first cell is a patient derived ovarian cancer cell; the second cell is a fibroblast, vascular endothelial cell, pericyte, or adipocyte; and the third cell is a ovarian cancer cell;
xxii) when the first cell is a patient derived ovarian epithelial cancer cell; the second cell is a fibroblast, vascular endothelial cell, pericyte, or adipocyte; and the third cell is an ovarian epithelial cancer cell;
xxiii) when the first cell is a patient derived pancreatic cancer cell; the second cell is a fibroblast, vascular endothelial cell, pericyte, or adipocyte; and the third cell is a pancreatic cancer cell;
xxiv) when the first cell is a patient derived uveal melanoma cell; the second cell is a fibroblast, vascular endothelial cell, pericyte, or adipocyte; and the third cell is a uveal melanoma cell.

Embodiment 26. A method for detecting a response of a patient-derived cancer cell to an anti-cancer therapeutic comprising:
culturing the triple co-culture according to any one of embodiments 1-25 in the presence of a test agent, wherein the test agent is an anti-cancer therapeutic or a candidate anti-cancer therapeutic; and
detecting the response to the test agent of the first cell, the second cell, and the third cell.

Embodiment 27. The method of embodiment 26, comprising: culturing the triple co-culture for a first time period;
administering to the co-culture the test agent at the conclusion of the first time period; and detecting the response to the test agent, of the first cell, the second cell, and the third cell after a second time period.

Embodiment 28. The method of embodiment 27, further comprising comparing the response to the test agent of the first cell to the response to the test agent of the second cell and the third cell.

Embodiment 29. The method of any one of embodiments 26-28, wherein the response comprises changes in cell growth, cell number, cell size, and/or cell morphology.

Embodiment 30. The method of any one of embodiments 27-29, wherein the second period comprises at least one cell cycle of the first cell.

Embodiment 31. The method of any one of embodiments 26-30, further comprising scoring the first cell as sensitive to the test agent if the first cell exhibits an adverse response after the second time period, and the second cell does not exhibit an adverse response after the second time period.

Embodiment 32. The method of embodiment 31, further comprising scoring the first cell as sensitive if the third cell exhibits an adverse response after the second time period.

Embodiment 33. The method of embodiment 31 or embodiment 32, wherein the adverse response comprises apoptosis, necrosis, an inhibition of cell growth, an inhibition of cell division, or an abnormal cell morphology.

Embodiment 34. The method of embodiment 32 or embodiment 33, wherein the second cell comprises a first reporter and the third cell comprises a second reporter, and wherein detecting the adverse response comprises detecting a loss of reporter activity.

Embodiment 35. The method of any one of embodiments 26-34, wherein detecting the response to the test agent comprises fluorescent activated cell sorting (FACS).

Embodiment 36. The method of any one of embodiments 27-35, wherein detecting the response to the test agent comprises FACS at the conclusion of the second time period.

Embodiment 37. A method for analyzing a set of test agents comprising:
culturing a set of triple co-cultures, wherein each triple co-culture of the set comprises the triple co-culture according to any one of embodiments 1-25, and wherein each triple co-culture in the set comprises the same patient derived cancer cell, and the same second cell and third cell;
administering to each triple co-culture of the set a different test agent from the set of test agents or a different concentration of the same test agent; and
detecting a response to the test agents of the first cell, the second cell, and the third cell in each triple co-culture.

Embodiment 38. The method of embodiment 37, comprising: culturing each triple co-culture for a first time period;
at the conclusion of the first time period, administering to each co-culture a test agent from the set of test agents or a different concentration of the same test agent; and
detecting the response to the test agent of the first cell, the second cell and the third cell in each co-culture after a second time period.

Embodiment 39. The method of embodiment 37 or embodiment 38, further comprising comparing the response to the test agent of the first cell to the response to the test agent of the second cell and the third cell.

Embodiment 40. The method of any one of embodiments 37-39, wherein the response comprises changes in cell growth, cell number, cell size, and/or cell morphology.

Embodiment 41. The method of any one of embodiments 38-40, wherein the second period comprises at least one cell cycle of the first cell.

Embodiment 42. The method of any one of embodiments 38-41, further comprising scoring the first cell as sensitive to the test agent if the first cell exhibits an adverse response after the second time period, and the second cell does not exhibit an adverse response after the second time period.

Embodiment 43. The method of embodiment 42, further comprising scoring the first cell as sensitive if the third cell exhibits an adverse response after the second time period.

Embodiment 44. The method of embodiment 42 or embodiment 43, wherein the adverse response comprises apoptosis, necrosis, an inhibition of cell growth, an inhibition of cell division, or an abnormal cell morphology.

Embodiment 45. The method of any one of embodiments 38-44, wherein the second cell comprises a first reporter and the third cell comprises a second reporter and detecting the adverse response comprises detecting a loss of reporter activity.

Embodiment 46. The method of any one of embodiments 37-45, wherein detecting the response to the test agent comprises FACS.

Embodiment 47. The method of any one of embodiments 37-46, wherein detecting the response to the test agent comprises FACS at the conclusion of the second time period.

Embodiment 48. A method for preparing a triple co-culture composition comprising:
a first cell, wherein the first cell is a patient-derived cancer cell;
a second cell, wherein the second cell is a non-tumor derived cell that supports growth and/or viability of the first cell; and
a third cell, wherein the third cell is a tumor-derived cell that is not derived from the patient and that exhibits sensitivity to at least one anti-cancer drug or drug combination,
wherein the first cell, second cell, and third cell are in a cellular aggregate;
the method comprising mixing the first cell, the second cell, and the third cell and facilitating the formation of the cellular aggregate.

Embodiment 49. The method of embodiment 48, wherein facilitating the formation of the cellular aggregate comprises embedding the first cell, the second cell, and the third cell with biologically functionalized magnetic nanoparticles that render the cells magnetic and applying an external magnetic field to assemble the magnetic first cell, the second cell, and the third cell into the cellular aggregate.

Embodiment 50. The method of embodiment 48, wherein facilitating the formation of the cellular aggregate comprises applying the first cell, the second cell, and the third cell into a container having a cell-repellent inner surface and incubating the cells for a sufficient period to allow the cells added to the container to coalesce into the cellular aggregate.

Embodiment 51. The method of any one of embodiments 48-50, comprising mixing the first cell, the second cell, and the third cell with a patient derived non-cancer cell from the patient's cancer microenvironment.

Embodiment 52. The method of embodiment 51, wherein the patient derived non-cancer cell from the patient's cancer microenvironment is a healthy stromal cell, an infiltrating immune cell, or an endothelial cell.

Embodiment 53. The method of any one of embodiments 26-52, wherein the method is performed in vitro.

### EXAMPLES

The following examples are put forth so as to provide those of ordinary skill in the art with a complete disclosure and description of how to make and use the embodiments, and are not intended to limit the scope of what the inventors regard as their invention, nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used.

### Example 1—Preparation of Tox Control Cells

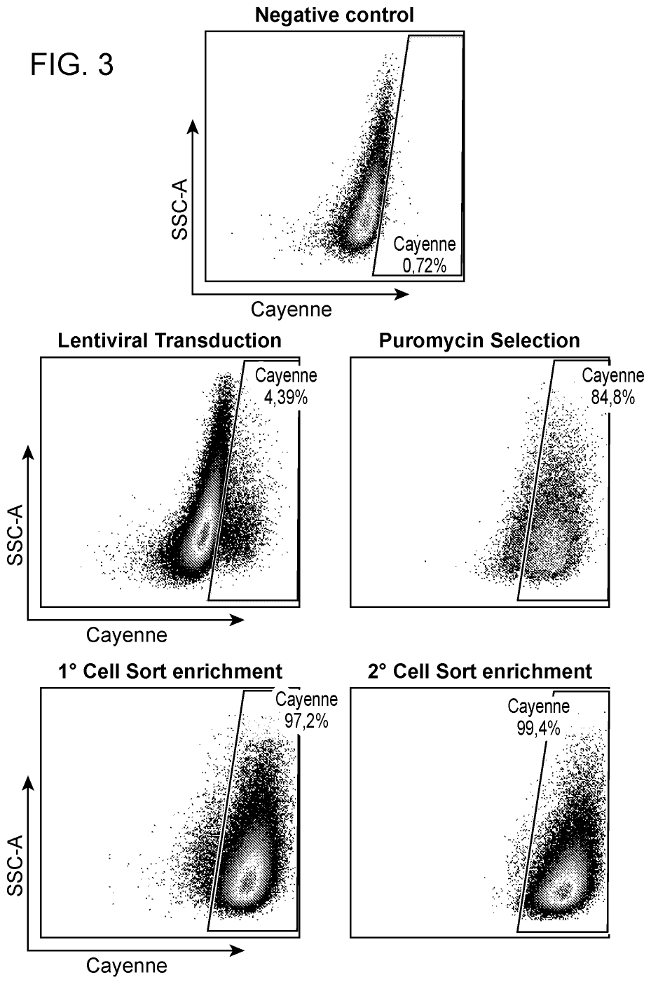

TC cells were prepared using a human bone marrow stroma cell line of clonal origin: HS-5 (ATCC® CRL-11882™). The cells were tagged by lentiviral transduction with a vector expressing a CMV-driven red fluorescent protein (RFP) variant (Cayenne) designed and assembled by ATUM Bio (Newark, California). The lentiviral vector also contains a puromycin resistance cassette. At 48 hours after transduction, puromycin (2 μg/ml—SIGMA) was added to DMEM culture media (Invitrogen) supplemented with 5% FBS (Internegocios FRA—Argentina). After 2 weeks of selection (about 4 passages), the cells were subjected to a primary cell sort enrichment by FACS, and cells with higher RFP expression (about 20% of the total cell population) were isolated. Later, puromycin selection was resumed, cells were re-sorted by FACS in a secondary cell sort enrichment, and expanded in culture for another 2 weeks to generate the working stocks, which were then stored in liquid nitrogen. The FACS plots obtained during this process are provided in FIG. 3.

### Example 2—Preparation of System Control Cells

SC cells were generated from a human leukemia cell line. Jurkat T-ALL cells (ATCC® TIB-152™) were tagged by lentiviral transduction with a vector expressing a CMV-driven green fluorescent protein (GFP) variant (Dasher) designed and assembled by ATUM Bio (Newark, California). This lentiviral vector also contains a puromycin resistance expression cassette. After 48 h after transduction, puromycin (4 μg/ml—SIGMA) was added to RPMI culture media (Invitrogen) supplemented with 10% FBS (Internegocios FRA—Argentina). After 2 weeks of selection (about 4 passages), cells were subjected to a primary cell sort enrichment by FACS, cells with higher GFP expression (about 20% of the total cell population) were isolated. Later, puromycin selection was resumed and a re-sorted by FACS in a secondary cell sort enrichment to select a subpopulation with higher brightness. These cells were expanded in culture to generate the working stocks, which were then stored in liquid nitrogen. The FACS plots obtained during this process are provided FIG. 4.

### Example 3—Preparation of PDC Cells

Samples of bone marrow or peripheral blood from patients diagnosed with ALL were subjected to two assays, which were performed directly on the fresh samples.

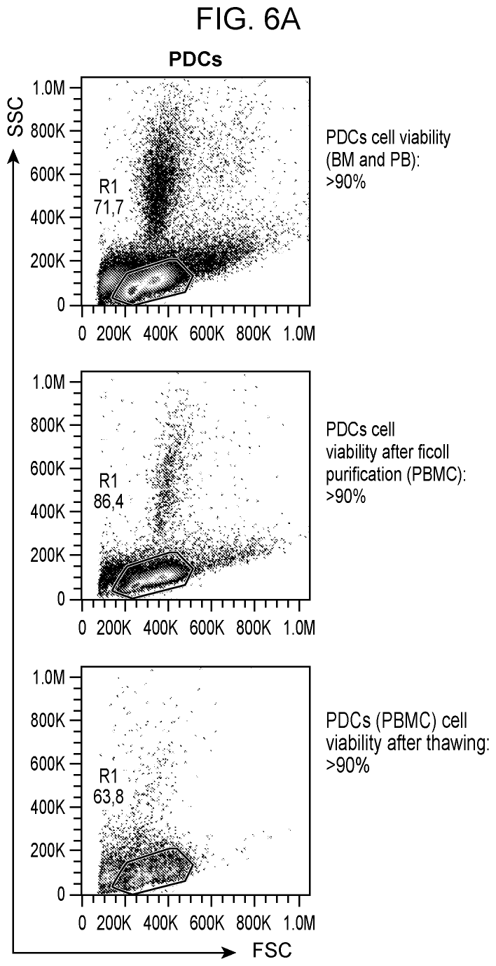

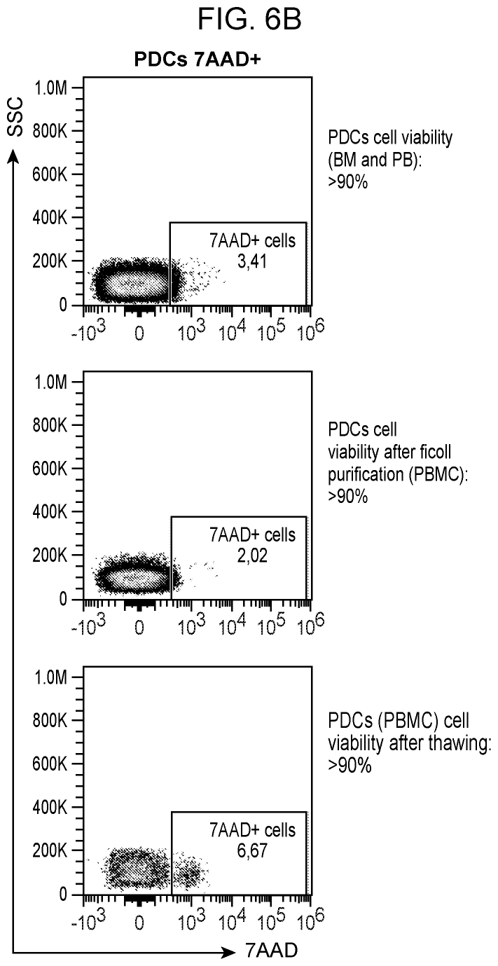

First, a survival assay using 7-aminoactinomycin D (7-AAD) was performed on a portion of the patient sample by FACS (Zembruski, et al. (2012) Anal. Biochem. 429, 79-81). Such quality control is typically used in samples that have been transported from a remote location. Only samples with a viable cell population greater than 90% of total cells are further processed. FIG. 6B, top panel, provides an example of results of cell viability analysis of cell viability tests.

Second, the percentage of pathological cells was determined by staining with anti-CD45 and anti-CD34 antibodies, and analysis by FACS. A cutoff of 20% was used as the minimal percentage of pathological cells to enter a purification procedure, either for immediate ex-vivo testing or for freezing (e.g., in 90% FBS/10% DMSO) and storage in the Biobank.

In the final steps of PDC preparation, patient samples, either fresh or frozen, were processed by a standard Ficoll-Paque protocol as described in Kizhakeyil, et al. (2019) Methods Mol. Biol. 1930, 11-17. Peripheral blood mononuclear cells (PBMCs) were isolated by aspiration from the gradients. A second viability control was performed using 7-AAD, and only samples with a viability greater than 90% are selected for use in further processing. FIG. 6B provides examples of the result of cell viability analysis of PBMCs after Ficoll purification of fresh (middle panel) or thawed (bottom panel) patient samples.

This final PBMC fraction is used as PDC cells in assembly of a triple co-culture. The PDC cells are placed in conditioned media obtained from HS-5 bone marrow/stroma cells. The media is supplemented with human autologous serum (5-20%) when available, otherwise, fetal bovine serum (5-10%) is used. At this step, cells can be counted and seeded into the co-culture assay and/or frozen in 90% FBS/10% DMSO for future use.

### Example 4—Co-Culture of TC Cells and SC Cells

FIGS. 5A-5D provides the results of co-culture of HS-5 cells as TC cells (prepared as in Example 1) and Jurkat cells as SC cells (prepared as in Example 2), and cytometric analyses of this co-culture. Different starting number of Dasher-Jurkat cells (5K, 10K, and 25K) were seeded in RMPI media 5% fetal bovine serum (FBS) in a 96-well plate format as mono-cultures or co-cultured with 6K Cayenne-HS-5 cells. At day 3 (FIG. 5A) or at day 7 (FIG. 5B), cells were analyzed by FACS and total number of Jurkat cells was determined for each experimental condition. The dot plots showing the co-culture at both endpoints are shown in FIGS. 5C and 5D.

### Example 5—Co-Culture of PDC Cells and Tc Cells

FIG. 7A provides the results of co-culture of PDC cells from an ALL patient prepared as in Example 3 and HS-5 cells as TC cells (prepared as in Example 1) and Jurkat cells as SC cells (prepared as in Example 2), and cytometric analyses of this co-culture. The top panel of FIG. 7A shows a series of FACS plots from analysis of PDC cells derived from acute lymphocytic leukemia (ALL) patients cultured in HS-5 conditioned media. The bottom panel of FIG. 7A shows a series of FACS plots from a dual co-culture of PDC cells derived from ALL patients with HS-5 cells. The increased survival of PDC cells is already observed in the initial gate (FSC vs. SSC) and the sequential analysis using anti-CD45 (pan leukocyte marker) and anti-CD34 (hematopoietic progenitor) to identify the patient derived cancer cells. An increased survival, in relative ratios and absolute numbers is observed for the patient derived cancer cells co-cultured with HS-5 cells. FIG. 7B shows an example of a triple co-culture with PDCs from an ALL patient. The gating strategy allows the rapid identification of system control (BL1; +Dasher-GFP), Tox control (BL3; +Cayenne-RFP) and PDC cells (negative for both fluorescent proteins). Whitin the PDCs gate, a sequential analysis of percentage of pathological cells is performed using anti-CD34 antibodies.

### Example 6—Assembly of Triple Co-Culture

FIG. 7B provides the results of a triple co-culture of PDC cells from an ALL patient prepared as in Example 3 and HS-5 cells as TC cells (prepared as in Example 1) and Jurkat cells as SC cells (prepared as in Example 2), and cytometric analyses of this co-culture. The gating strategy allows the rapid identification of system control (BL1; +Dasher-GFP), Tox control (BL3; +Cayenne-RFP) and PDC cells (negative for both fluorescent proteins). Whitin the PDCs gate, a sequential analysis of percentage of pathological cells is performed using anti-CD34 antibodies.

At day 0, 5K-10K TC (Tox Control) cells (e.g., prepared according to Example 1) were seeded in each well of a 96 multiwell plate in 100 μl of DMEM (GIBCO-US) supplemented with 5% FBS (Internegocios-Argentina).

At day 1, SC (System Control) cells (e.g., as prepared according to Example 2) and PDC cells (e.g., as prepared according to Example 3) were added in a ratio selected from 1:5 to 1:9 (e.g., about 5K-10K SC cells/well and about 25K PDC cells/well in a 96 well plate) using 25 μl/well of RPMI (GIBCO) supplemented with human autologous serum (5-20%) from the same patient when available. Otherwise, fetal bovine serum (5-10%) was used.

At day 2, the triple co-culture was ready for use in an assay.

FIG. 7C provide the results of culturing PDC cells ex vivo for six days as a single suspension (mono-culture) and in a triple co-culture system. 50,000 PDC cells were cultured ex vivo for six days either as a single suspension (mono-culture) in RPMI containing 10% FBS or in a triple co-culture system using the same medium. TC cells, SC cells, and PDC cells were used in the ratio of 1:1:10, respectively.

As shown in FIG. 7C, as compared to a mono-culture of PDC cells, the triple co-culture system increased the ex vivo survival of the PDC cells by six fold with PDC cells from an ALL patient and 60 fold with PDC cells from an AML patient.

### Example 7—Screening Anti-Cancer Drugs for Anti-Cancer Activity

The following is an example of a Drug Activity and Resistance Test (DART) as it can be applied for screening PDC cells from an ALL patient. FIGS. 8A-B provides an example of a triple co-culture, and a sample time for the ex vivo screening of patient cancer cells and analysis to identify an anti-cancer therapy options for a patient.

A triple co-culture system as illustrated in FIG. 8A is prepared as set out in Example 6 in a 96 well plate. In this example, the patient has been diagnosed with ALL, and the PDC cells used in the triple co-culture are obtained from a blood sample or bone marrow sample from the patient as described above. Tagged HS-5 bone marrow/stroma cell line, which are genetically modified to express a red fluorescent protein (RFP) variant (Cayenne) and exhibit puromycin resistance (as described in Example 1 above), serves as a Tox Control (TC) cell (“Cayenne Tox Control”). Tagged Jurkat T-ALL cells, genetically modified to green fluorescent protein (GFP) variant (Dasher) and puromycin resistance (as described in Example 2), serve as a System Control (SC) cell (“Dasher System Control”).

As illustrated in FIG. 8B, at day 0 the Cayenne Tox Control cells are seeded in each well of a 96 well plate (e.g., at about 5K-10K cells/well in 100 ul of DMEM (GIBCO-US) supplemented with 5% FBS), and incubated for about 1 day. At day 1, Dasher System Control cells and PDC cells are added in a ratio selected from 1:5 to 1:10 (e.g., about 5K-10K Dasher System Control cells/well and about 25K PDC cells/well) using 25 ul/well of RPMI (GIBCO) supplemented with either human autologous serum (5-20%) from the same patient as the PDC cells or with fetal bovine serum (5-10%). After another day of incubation, the co-culture system is ready for use (at day 2).

Wells are pre-designated as control wells (no drug), or for testing a “vulnerability dose”, a “resistance dose” and drug combinations. The selection of the doses of FDA approved drugs can involve the test of two reference doses for each drug and one optimal combination for each set of drugs. Such doses are defined on a case-by-case basis from a combination of patient database analysis from published works, and experimental data obtained internally using a wide variety of dose-response schemes with multiple different PDC cell samples. A low and high dose for each drug in the matrix is defined. Since only extremely sensitive patients show ex vivo sensitivity at very low doses, the goal of such very low doses is to uncover clear vulnerabilities in patients (“vulnerability proxy” or “vulnerability dose”). In contrast, the goal of the high doses is to identify those patients that are highly likely to be resistant to that drug in vivo (“resistance proxy” or “resistance dose”). This combination of positive and negative predictive power provides valuable insights in clinical settings for oncology therapy selection, both at initial and subsequent lines of treatment.

The drug (or drug combination) of interest is added to the test wells. Drugs can be obtained from screening libraries, such as the anti-cancer approved drug Library (TargetMol—US). Examples of drugs and drug combinations suitable for screening of PDC cells from an ALL patient is provided in FIG. 8C.

Even though tissue culture plates are incubated in a humidified CO₂ incubator (37° C.; 5% CO₂), evaporation can occur during long-term screening experiments (e.g., over 3 days, 5 days, 7 days, or longer. To counteract this undesirable phenomenon, PBS is added in the interstitial space between the wells of the multiwell plate. With this setting, a 7-day incubation period can maintain about ⅔ of the total initial volume in a 96 well plate. In case of using 384 well plates, all volumes and cell numbers are decreased by about a 1:4 ratio.

At about 6-7 days after treatment with the drug(s), the plates are analyzed by high-throughput flow cytometry (e.g., using an Attune NxT+Autosampler—Thermo Fisher Scientific). To detach adherent cells, standard enzymatic (i.e. Trypsin) and/or chemical (i.e. EDTA) procedures are used. At this step, the co-culture mix can also be fixated for future analysis using Paraformaldehyde 2%.

The gating strategy to identify and quantify the different cell types of interest in the triple co-culture by flow cytometry involves a series of steps with the goal of detecting the differential survival of malignant PDC cells.

An initial (optional) step is to perform an exclusion gate using a viability dye such as Sitox-Red, Ghost violet, DAPI, VivaFix or Annexin-V conjugated to various dyes. The negative (live) cells then enter a sequential gating strategy.

Initially an FSC/SSC plot is analyzed to identify the cell populations of interest and to discard cell debris and remaining dead cells. Subsequently, a gate is performed in the visible red channel (about 580 nM: blue laser) to quantify the Cayenne TC cells. The negative cells are then gated in the visible green channel (about 520 nM: blue laser) to quantify the Dasher SC cells.

The double negative (Cayenne negative, Dasher negative) cells are then gated using a different laser (e.g., red laser; APC-Cy7 channel) to identify the CD45⁺ cell population stained with a dye-conjugated anti-CD45 antibody. This gate allows the identification of the leukocyte fraction of the PDC cells. The CD45-low population is then sequentially gated to identify and quantify the CD34⁺ malignant cells in a different channel of the same laser (e.g., APC) using another specific dye-conjugated antibody (Biolegend—US). The specific toxicity of the treatments against PDC cells is then calculated by referring their effect to the response of the TC cells.

### Example 8—Identification of a Cancer Drug for Individualized Treatment

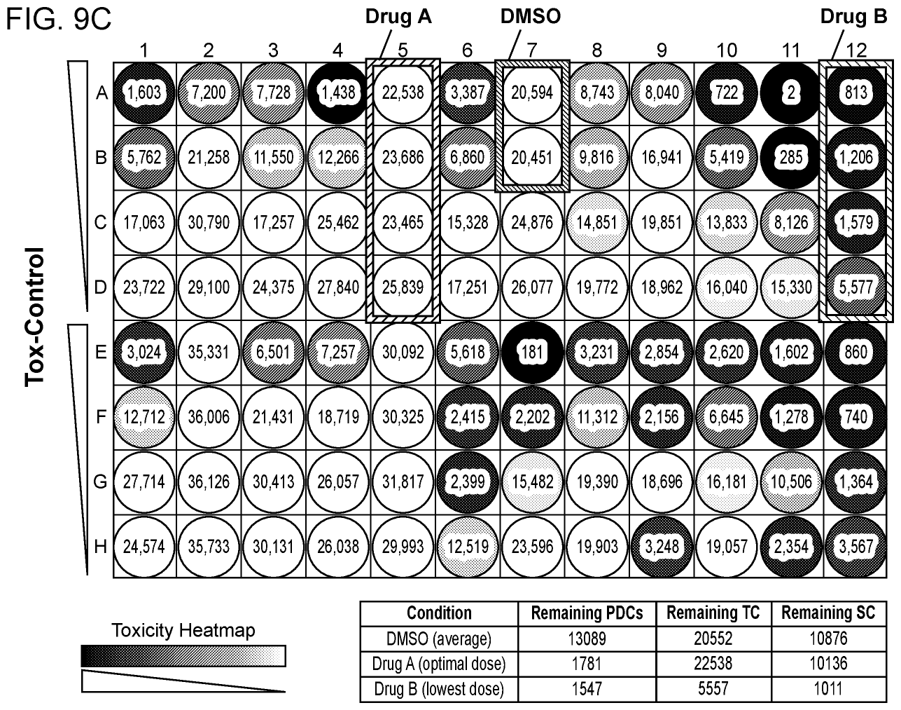

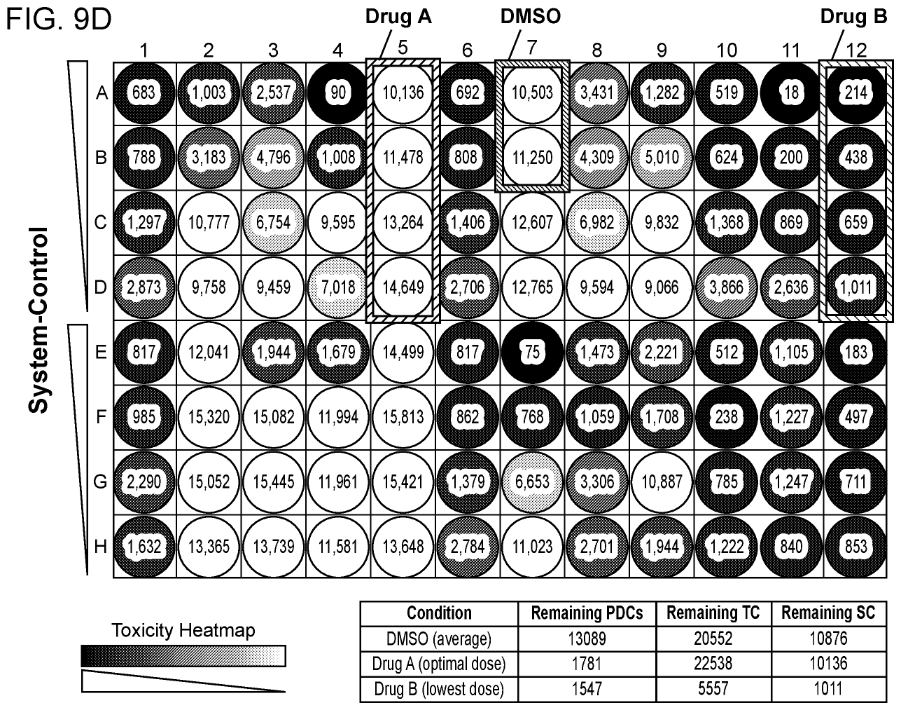

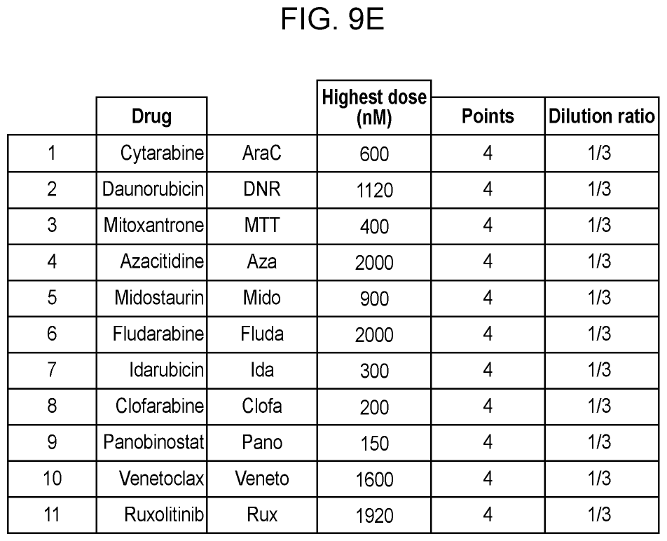

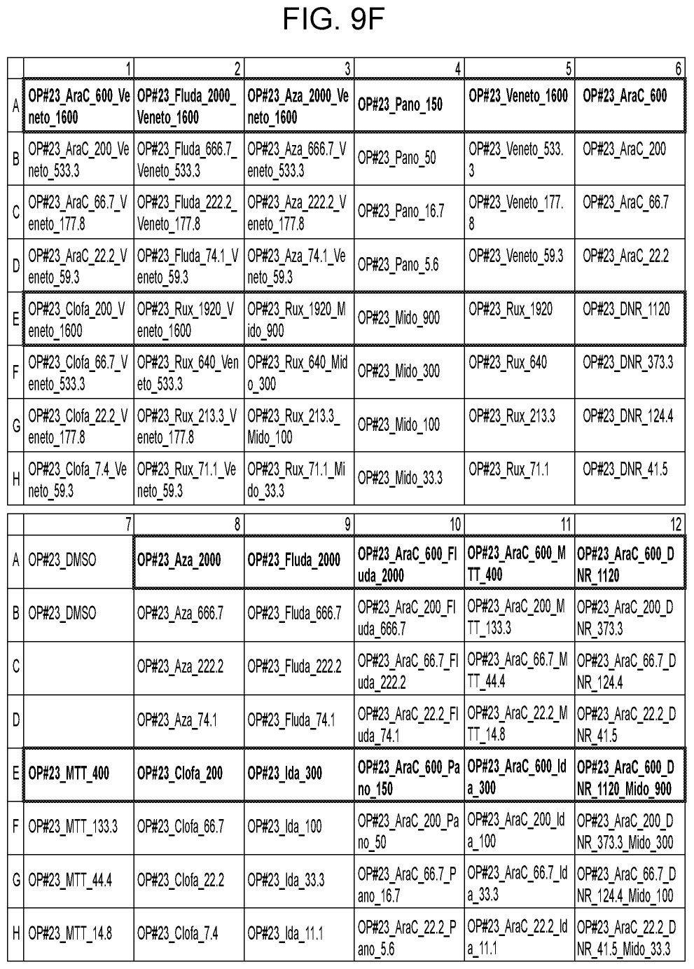

PDCs from patient samples from a biobank were screened for response to three different drugs. For the data presented in FIG. 9A, the PDC cells were obtained from a patient arbitrarily identified as OP #23. This patient had acute myeloid leukemia.

Triple co-culture systems using PDC cells from OP #23 were established as described in Examples 1-3. TC cells were derived from the human bone marrow stroma cell line HS-5. Cells were tagged by lentiviral transduction with a vector expressing a CMV-driven red fluorescent protein (RFP) variant (Cayenne), which also contains a puromycin resistance cassette. After 2 weeks of puromycin selection, cells were enriched by cell sorting using FACS. The enriched population was submitted to puromycin selection for two additional weeks and cells were re-sorted by FACS to achieve a purity higher than 99%. SC cells were derived from the human myelogenous leukemia cell line K-562. Cells were tagged by lentiviral transduction with a vector expressing a CMV-driven green fluorescent protein (GFP) variant (Dasher), which also contains a puromycin resistance cassette. After 2 weeks of puromycin selection, cells were subjected to enrichment by cell sorting using FACS. The enriched population was submitted to puromycin selection for two additional weeks and cells were re-sorted by FACS to achieve a purity higher than 99%. The PDC cells sample were obtained from a bone marrow biopsy. Sample viability QC was performed using 7-aminoactinomycin D (7-AAD) before and after processing by Ficoll-Paque to obtain viable peripheral blood mononuclear cells (PBMCs) were isolated by aspiration from the gradients. This final PBMC fraction was used in the assembly of the triple co-culture.

In the triple co-cultures, 50,000 PDC cells were mixed with 5,000 TC Cells and 5,000 SC cells. Thus, the ratio of the PDC cells: SC cells: TC cells was 10:1:1. The mixture of cells was incubated for 1 day to establish the triple co-culture.

The triple co-cultures were then subjected to four treatments: 1) Control wells with the vehicle of the drugs, e.g., DMSO; 2) Panobinostat at a concentration of 5 nM; 3) a combination of Cytarabine at 22 nM and Daunorubicine at 41 nM; and 4) Venetoclax at a concentration of 1.6 μM. The triple co-cultures were incubated for 6 days for the treatments to exert the effects on the cells.

After a treatment period of 6 days, cells from the triple co-cultures were examined using multiparametric flow cytometry. TC cells were identified by the expression of the red fluorescent protein Cayenne; SC cells were identified by the expression of the green fluorescent protein Dasher; and PDC cells were identified by their lack of fluorescence.

As shown in the top left panel of FIG. 9A, the control wells, i.e., wells mock-treated with the vehicle of the drugs, show the reference number of PDC cells, SC cells, and TC cells in the co-culture.

The top right panel in FIG. 9A shows a typical inactive treatment. Here, the triple co-culture was treated with Panobinostat, which showed a mild effect on the SC cells; however, neither the PDC cells nor the TC cells showed a response. Such lack of cytotoxic activity on the PDC cells, i.e., inactive treatment, is frequently observed, most commonly at the vulnerability doses of most treatments.

The bottom left panel in FIG. 9A shows a typical active but toxic response. This is another frequent response observed, most commonly at the resistance dose of chemotherapeutic treatments. Here, the treatment shows activity with high concomitant toxicity. As shown in the bottom left panel of FIG. 9A, the combination of Cytarabine and Daunorubicine showed such active but toxic response. In this treatment, a strong cytotoxic effect was observed in the SC cells, but most importantly also in the PDC cells with a concomitant strong cytotoxic effect in the TC cells.

Finally, the bottom right panel in FIG. 9A shows a typical active plus selective response. This is a less frequent, yet highly desirable outcome and provides for the selective activity of a treatment against the PDC cells. In this type of response, cytotoxic effects can be observed in the SC cells, but such response should be negligible in the TC cells. An example of such active treatment is provided by Venetoclax, which is shown in the bottom right panel of FIG. 9A. As shown in the bottom right panel of FIG. 9A, a strong cytotoxic effect was also observed in the PDC cells, while no cytotoxic effect was observed in the TC cells.

Thus, FIG. 9A shows that for patient OP #23, the DART prediction is that Venetoclax would provide a desirable therapy, i.e., strong therapeutic effect with minimal to no side effects.

### Example 9—Screening a Panel of Cancer Drugs for Individualized Treatment

A panel of 23 cancer treatments was screened for desirable therapeutic activity on the cells from the patient OP #23.

Triple co-cultures containing PDC cells from the patient OP #23 were established as described in Example 8.

Triple co-cultures containing 50,000 PDC cells, 5,000 TC cells, and 5,000 SC cells were established in each well of a 96 well plate. After incubating the triple co-cultures for 6 days with different treatments, the cells were analyzed by multiparametric FACS.

Twenty-three drugs were tested, each with four doses. The drugs used and their respective concentrations are provided in FIGS. 9E-9F. DMSO treated wells were used as controls.

After incubation with the drugs for 6 days, the cells from the triple co-cultures were examined by flow cytometry as described in Example 8. The effects of the 23 drug treatments with four-point dose response on the cells from patient OP #23 are shown in FIG. 9B as a heatmap of a 96-well plate in grayscale. Two replicas of the untreated (DMSO) controls are shown in FIG. 9B. Venetoclax depicts a selective activity against the PDC cells, showing no cytotoxic activity on the TC cells (FIG. 9C). A combination of Cytarabine and Daunorubicine shows an active and toxic response, strongly affecting the PDC cells but also affecting the TC cells (FIG. 9C) and SC cells (FIG. 9D). The tables shown at the bottom of FIGS. 9B, 9C, and 9D summarize the number of remaining viable cells for PDC cells, TC cells, and SC cells upon treatments with Venetoclax and combination of Cytarabine and Daunorubicine.

FIGS. 9B-9D show that for patient OP #23, Venetoclax would provide a desirable “active and selective” therapy, i.e., strong therapeutic effect with minimal to no side effects. Also, the combination of Cytarabine and Venetoclax would provide “active but toxic” therapy, i.e., therapeutic effect but with possible severe adverse side effects.

### Example 10—Estimating a Therapeutic Effects of Cancer Drugs for Individualized Treatment

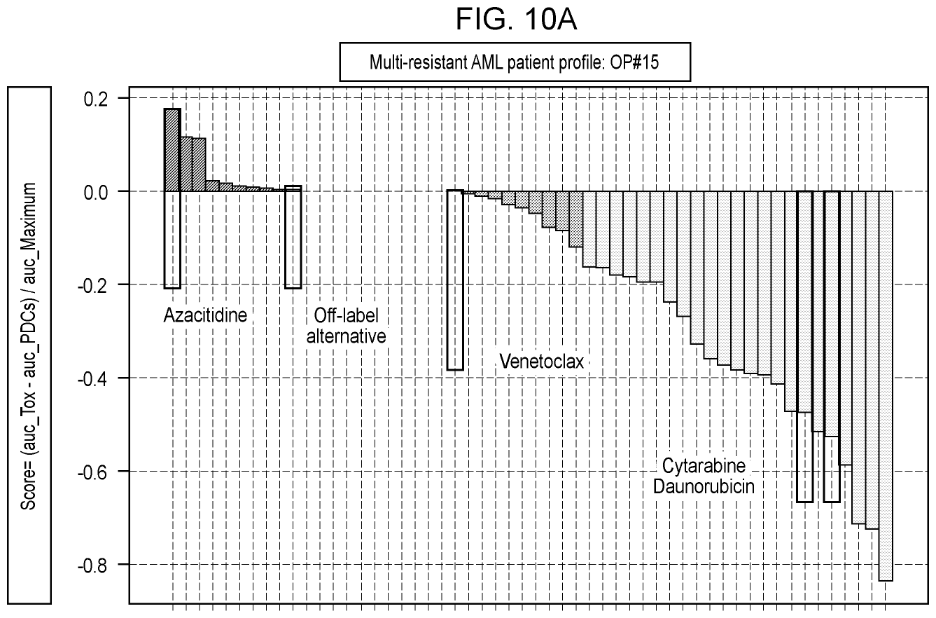

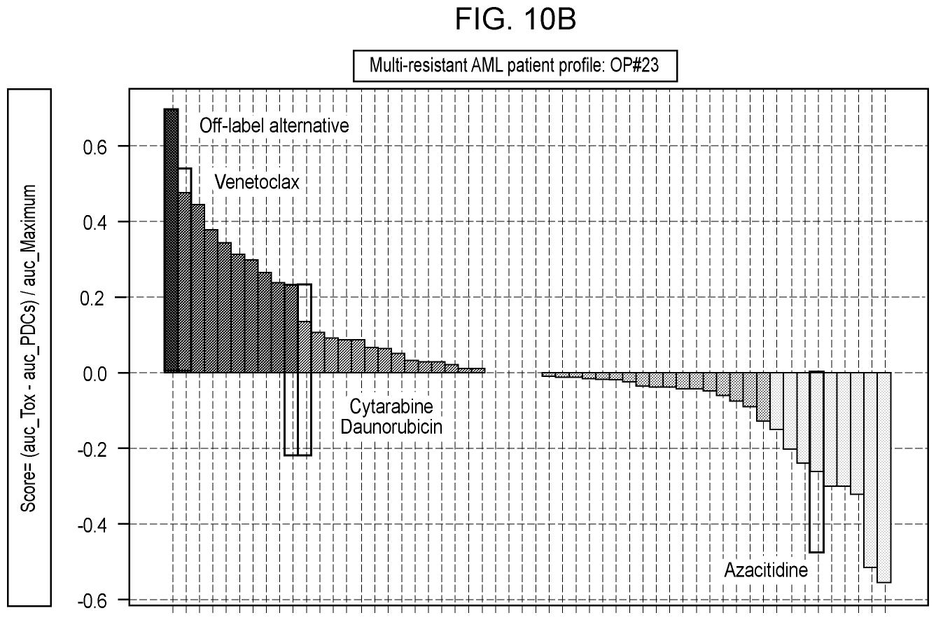

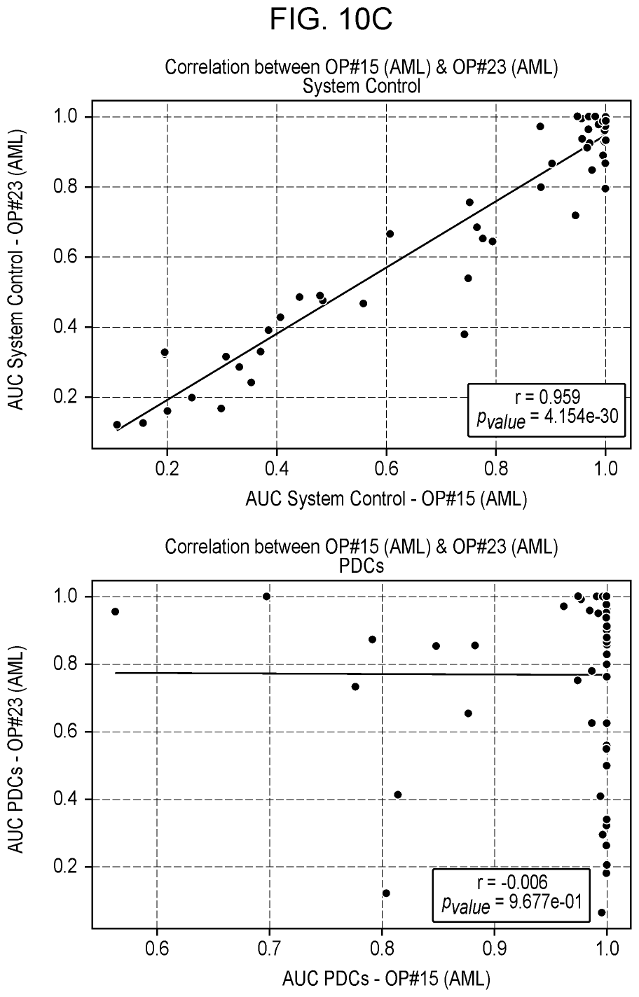

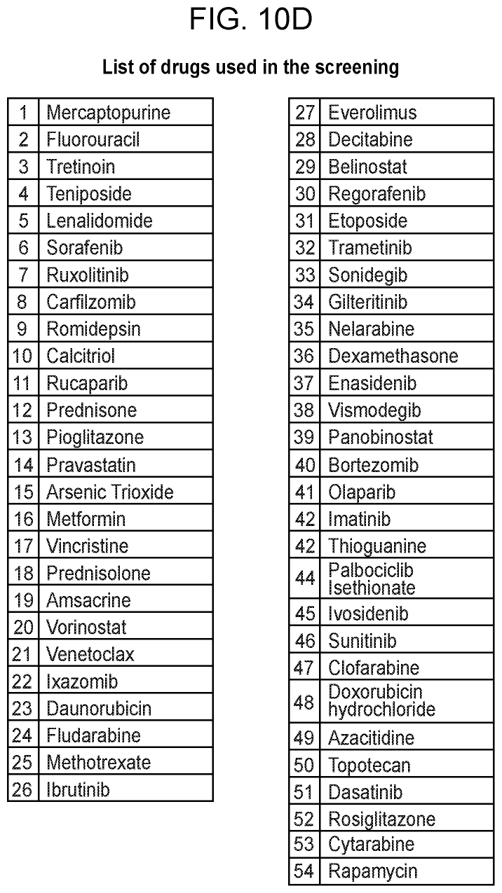

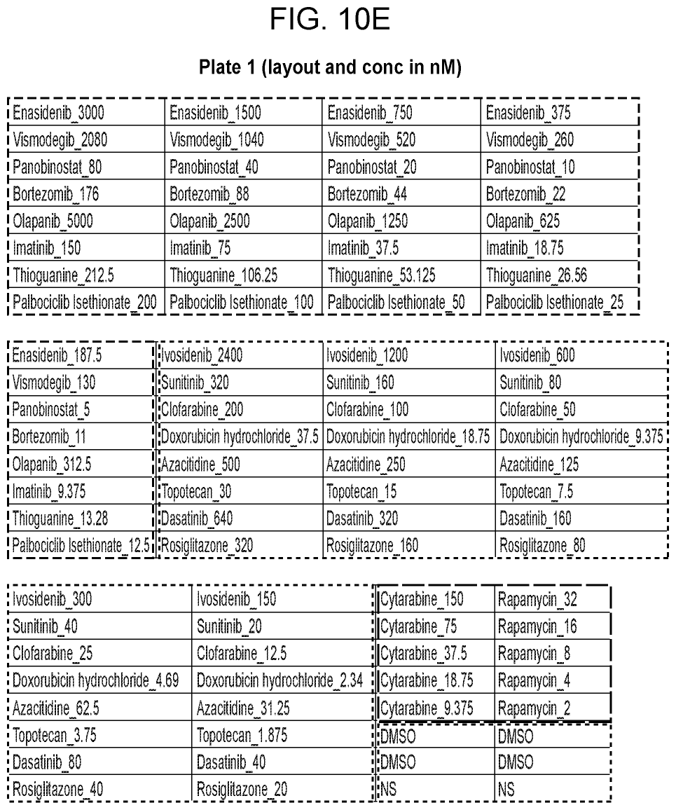

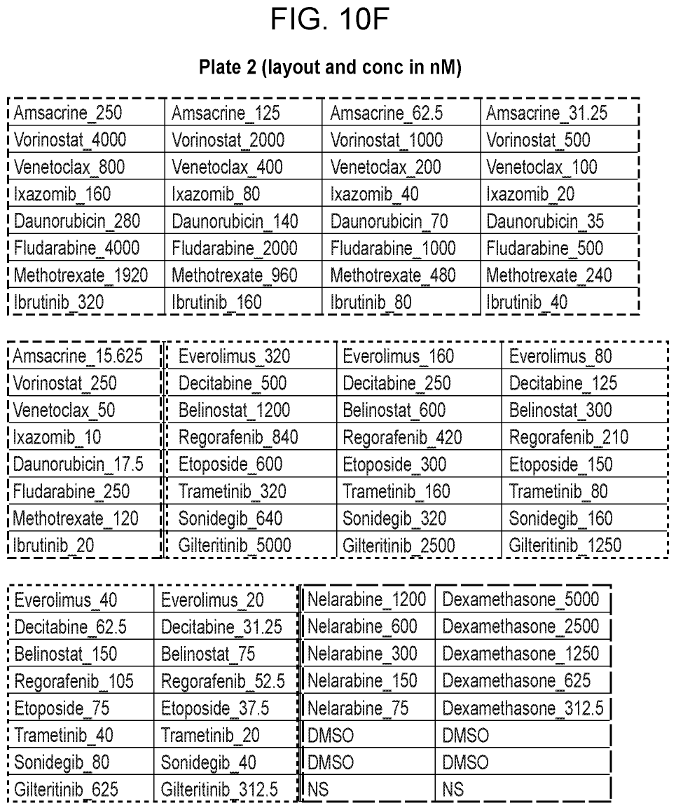

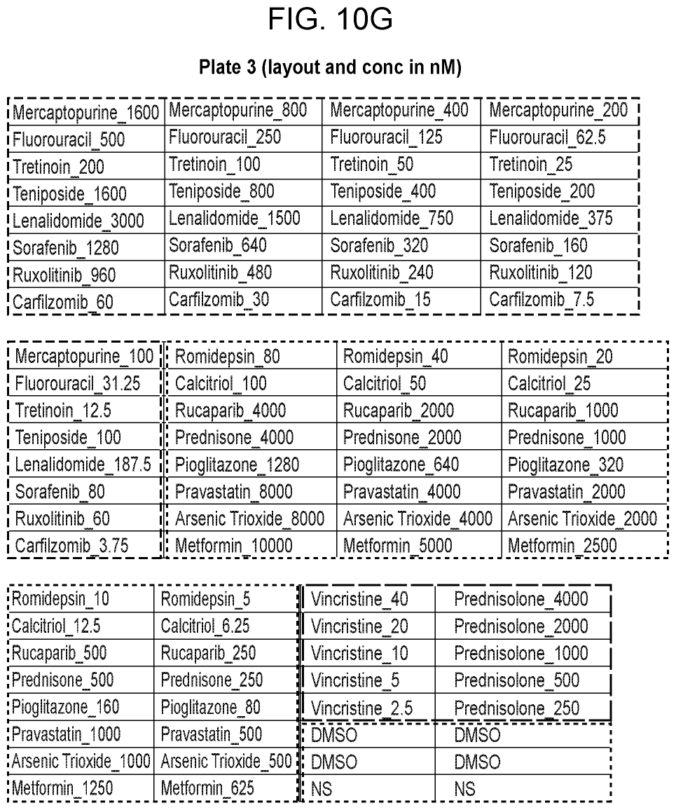

Triple co-culture system was used to assess the response of cancer cells obtained from a biobank from two patients (OP #15 and OP #23). Triple co-cultures containing the PDC cells from the patients were established as described in Example 8. PDC cells in the triple co-cultures were exposed to fifty-four mono-drugs, each in a 4-point dose-response. The four-point dose responses covered resistance and vulnerability doses for each tested drug. The drugs used and their respective concentrations are provided in FIGS. 10D-10G. DMSO treated wells were used as controls.

The PDC cells from the two patients exhibited varied response to various tested drugs as well as varied pattern of response. The data is presented in FIG. 10A and FIG. 10B and is described below.

Patient OP #15 showed a multi-resistant phenotypic response and patient OP #23 showed a multi-sensitive response. PDC cells from patient OP #15 were resistant to multiple cancer therapeutics, whereas PDC cells from patient OP #23 were sensitive to multiple cancer therapeutics.

The sensitivity/resistance to a particular therapeutic was assessed based on a scoring procedure that leverages the Areas Under the Curve (AUCs) for each drug in the PDC cells and TC cells. The AUC is calculated by the area below the curved lined formed by the percentage of survival (in comparison to DMSO) of the four points of the dose-response. The AUC maximum is the area obtained when the survival at the four points of dose-response is 100%. Following formula was used to determine the score.

$$Score = ( AUC\_Tox - AUC\_PDCs ) / AUC\_Maximum$$

For a particular drug, a score above 0 indicates increased activity against the PDC cells (selectivity), while a score below 0 indicates higher toxicity and poor selectivity. The rectangles highlight the standard of care received by these patients (Cytarabine and Daunorubicin) and the top predictions of the tested drugs for both patients. The top tested drug for OP #15 is azacitidine attributed to the highest score above 0. As indicated by the highest scores of above 0 for these drugs, the top tested drugs for OP #23 are venetoclax and an off-label drug, which is a tyrosine kinase inhibitor used for the treatment of other hematological malignancy.

### Example 11—Triple Co-Culture System Provides Consistent Effects for Different Drugs on Sc Cells

In the triple co-culture systems disclosed herein, the SC cells provide an internal positive control for the test agent being assayed. For example, the SC cells have a known sensitivity or resistance to the test agent being assayed. Therefore, when two triple co-culture systems are developed with the same SC cells but with different PDC cells, the effects of a tested therapeutic on the SC cells act as a proxy of the proper operation of the assay. In other words, in appropriately operational triple co-culture systems containing specific SC cells but different PDC cells, a tested therapeutic should exert similar effects on the SC cells. Therefore, the effects on the SC cells of a number of screen therapeutic agents could be used to assess the impact of experimental variables, such as PDC cells, culture system, media, FBS, culture conditions, drugs, etc. on the operation of the triple co-culture system.

The impact of the experimental variables on the effect of various therapeutics on SC cells was assessed. The data is presented in FIG. 10C, which shows drug-to-drug correlations for the effects on the SC cells in the screening assay performed for the patients depicted in FIGS. 10A and 10B. The high correlation observed in the SC cells in the response to the 54 drugs of the matrix confirmed that all the experimental variables (triple co-culture system, media, FBS, culture conditions, drugs, etc.) are comparable between the patients.

In contrast to the effects observed on the SC cells, the low correlation between the two patients in the effect of the therapeutics on the PDC cells indicates a remarkably different response of the PDC cells to the different drugs tested. In other words, for several drugs, the effects on the PDC cells from patient OP #23 were substantially different from the effects of the same drug on the PDC cells from patient OP #15.

These data show that the triple co-culture system provides consistent effects on PDC cells throughout a variety of therapeutics regardless of the experimental variables and that the response of the SC cells acts as a robust internal control to determine which assays are reliable for predictions. These data also show that the triple co-culture system could be used to identify suitable therapeutic agents that would be expected to be effective in individualized patients.

### Example 12—Comparison of the Effects on PDC Cells of the Therapeutic Drugs Identified Using the Triple Co-Culture System and Standard Care

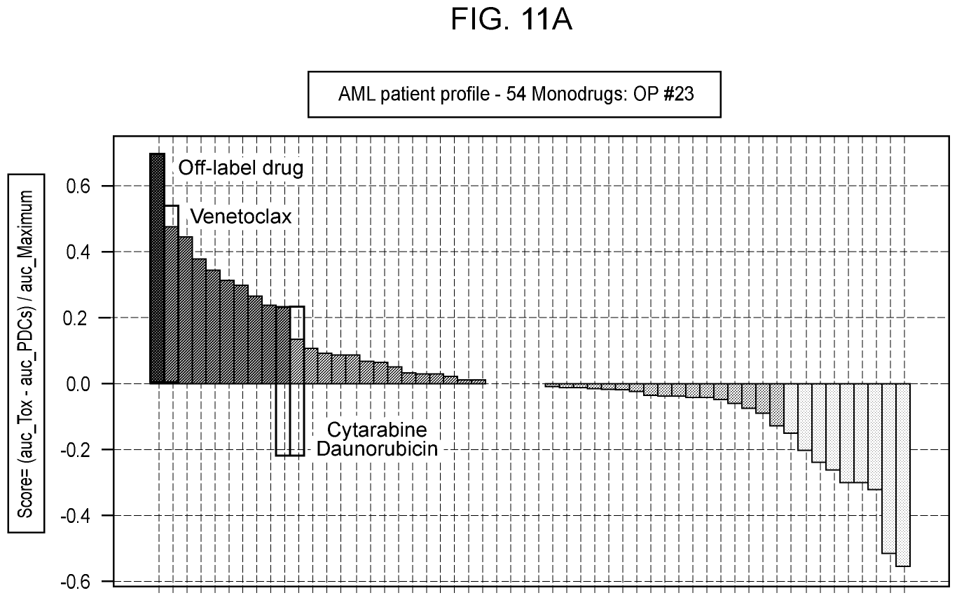

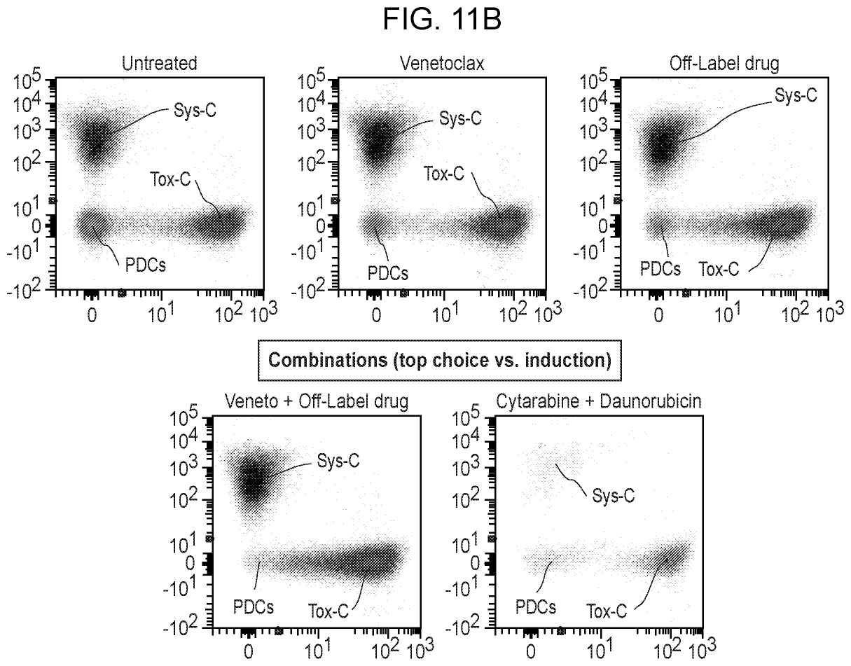

The drugs identified from the 54 monodrugs that would be expected to be therapeutically effective for patient OP #23 (FIG. 11A) were further tested for synergy and compared with the standard care. A combination of Cytarabine and Daunorubicin is one of most common standard care schemes used in the clinic and, therefore, this combination was used for comparison.

FIG. 11B shows the effects of the top performing monodrugs and drug combinations identified in the initial assay on the PDC cells from the selected patients. For patient OP #23, the two top performing drugs (Venetoclax+1 off-label drug) were combined at the lowest dose to seek additive/synergic effects. The combination of Cytarabine and Daunorubicin was tested in parallel as a standard care treatment. The results are expressed in FIG. 11B.

Initially, FIG. 11A provides varied response to various tested drugs on the PDC cells from patient OP #23. FIG. 11B provides flow cytometry data indicating the effects on the PDC cells, TC cells, and SC cells of the top performing drugs, namely, Venetoclax and an off-label drug, either individually or in combination. FIG. 11B also provides flow cytometry data indicating the effects on the PDC cells, TC cells, and SC cells of the combination of Cytarabine and Daunorubicin.

The flow cytometry data indicate that venetoclax and the off-label drug trigger cytotoxic effects on the PDC cells, while both drugs exerted effects on the SC cells and TC cells. The combination of venetoclax and the off-label drug exhibited strong cytotoxicity on the PDC cells but minimal effects on the TC cells.

The flow cytometry data also indicate that the combination of cytarabine and daunorubicin exhibited strong cytotoxic effects on the PDC cells as well as the TC cells. Therefore, the data from FIGS. 11A-11B suggest that the combination of venetoclax and the off-label drug, which are the desirable therapeutics identified using the triple co-culture system, would provide better therapeutic outcome with minimal side effects when compared to the standard of care treatment of the combination of cytarabine and daunorubicin.

### Example 13—Triple Co-Culture System in 3D Aggregates

Triple co-culture compositions were assembled into 3D cellular arrangements, such as cellular aggregates or spheroids. These 3D cellular arrangements are referenced in this Example as tumoroids.

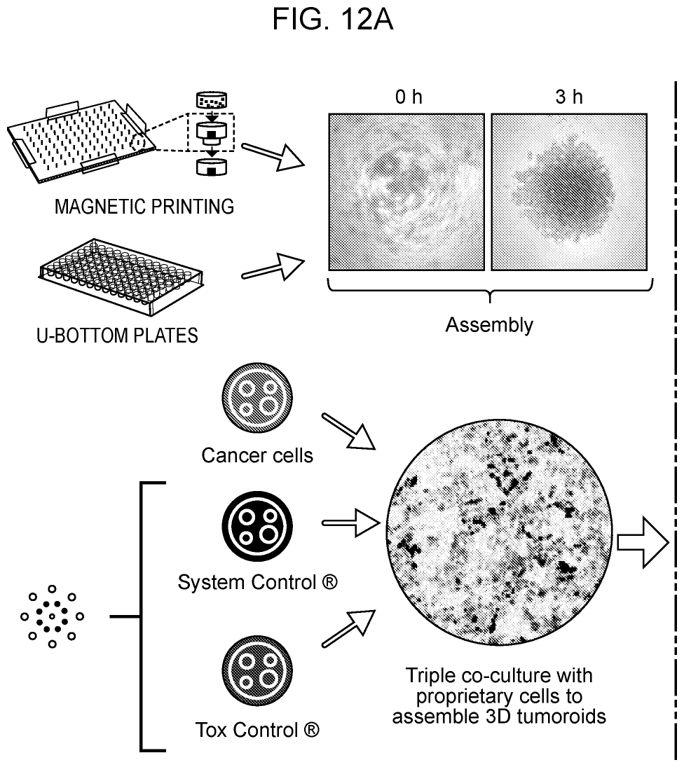

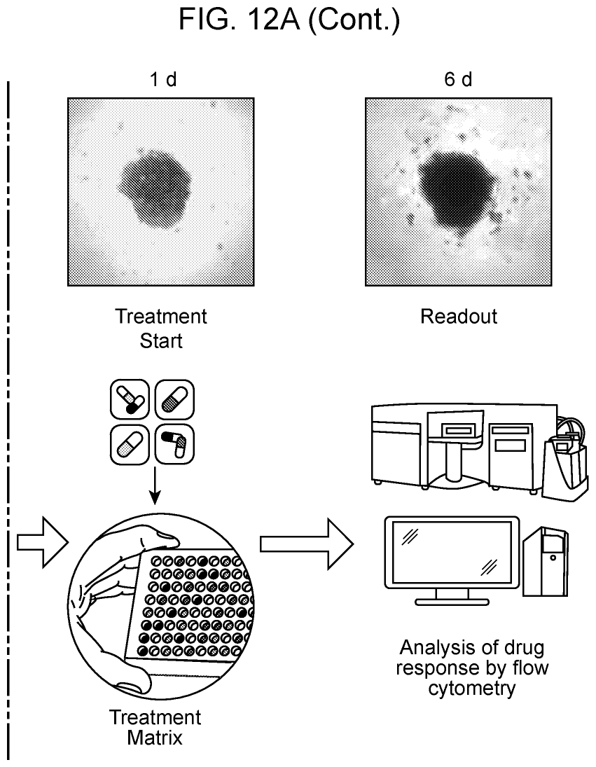

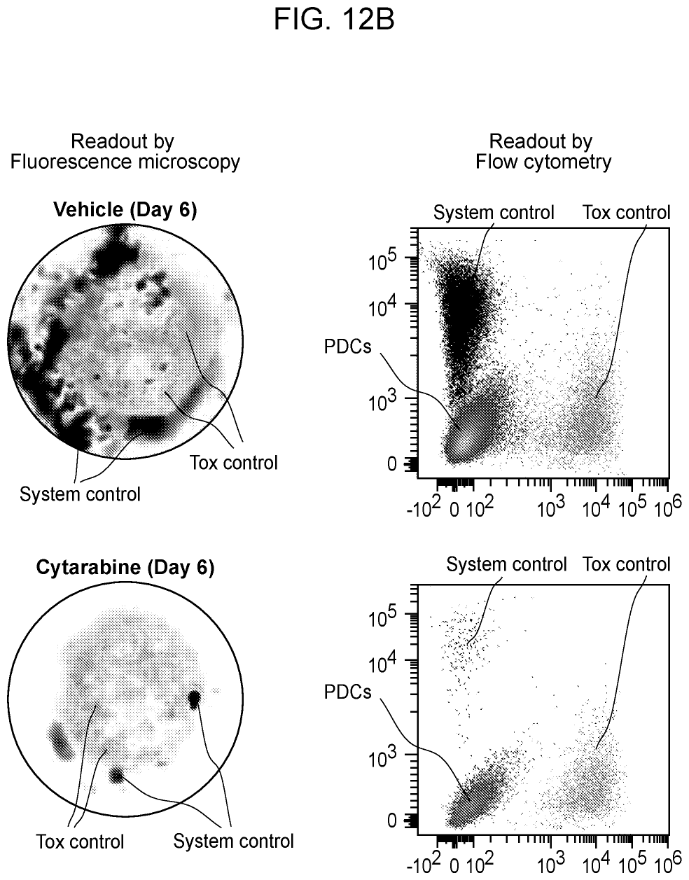

The 3D tumoroid was formed with or without extracellular matrix (ECM) embedded in culture media. FIG. 12A shows how monocellular suspensions containing PDC cells, SC cells, and TC cells at different ratios were incubated in culture media containing low percentages of ECM (0.5-5%) and tumoroids were assembled using magnetic printing or cell-repellent U-bottom plates with centrifugation. On day 1, when the tumoroids were in a fully compact 3D configuration, the multiwell plates containing the tumoroid replicas (1 per well) were subjected to a matrix of treatments, including mono-drugs and combinations. On day 6, the differential survival of PDC cells, TC cells, and SC cells was analyzed by flow cytometry using the methods described in Example 8.

### Example 13—Magnetic Bioprinting to Prepare Triple Co-Culture System Containing 3D Tumoroids

Magnetically printed 3D patient-micro-avatars (PMAs) using PDCs from an AML patient were prepared. PDCs from a bone marrow aspirate were processed by Ficoll-Paque to isolate the PBMC fraction, which was printed into 3D tumoroids in co-culture with SC cells and TC cells. Particularly, 10,000 PDC cells were mixed with 3,000 SC cells, and 8,000 TC cells.

3D tumoroids so formed were treated for 6 days with vehicle or Cytarabine at 200 nM. FIG. 12B shows vehicle treated and Cytarabine treated 3D tumoroids after 6 days of treatment. At the end of the treatment, tumoroids were analyzed first by florescence microscopy (left panel of FIG. 12B). The tumoroids were subsequentially disaggregated mechanically and analyzed by flow cytometry to quantify the surviving fraction of PDCs, SC cells, and TC cells (right panel of FIG. 12B). As shown in the flow cytometry results in FIG. 12B, cytarabine treatment exhibited strong cytotoxic effects on the cancer cells and SC cells and moderate cytotoxicity on the TC cells. These data also show that triple co-culture systems embedded into 3D tumoroids can also be used to screen cancer therapeutics.

### Example 14—Preparation of 3D Patient Micro-Avatar

3D cellular arrangements that mimic tumor microenvironment of a patient can be used to screen cancer therapeutics to identify their efficacy on cancer cells in their microenvironment. This is achieved by preparing 3D “patient micro-avatar” (PMA). Exemplary schematic representation of producing PMAs is provided in FIG. 13.

To prepare a 3D PMA, a biopsy from a patient's tumor is obtained and a suspension of cells from the biopsied sample is produced. Such suspension contains PDC cells as well as other non-cancerous cells, such as healthy stromal cells, infiltrating immune cells, endothelial cells, and other patient cells present in the tumor microenvironment of the patient. This cell suspension is then mixed with SC cells and TC cells to produce triple co-culture containing PMA.

The 3D cellular arrangement having PDC cells, patient's other cells, SC cells, and TC cells is performed using magnetic bioprinting or cell-repellent U-bottom plates with or without ECM components. The 3D PMAs so produced are treated with one or more cancer therapeutics and the cells from the PMA are studied by flow cytometry to identify PDC cells, SC cells, TC cells as well as other patient cells.

An effective cancer therapeutic would trigger cytotoxicity on the PDC cells while having minimal effects on the TC cells as well as other healthy patient cells.

While the present invention has been described with reference to the specific embodiments thereof, it should be understood by those skilled in the art that various changes may be made, and equivalents may be substituted without departing from the true spirit and scope of the invention. In addition, many modifications may be made to adapt a particular situation, material, composition of matter, process, process step or steps, to the objective, spirit, and scope of the present invention. All such modifications are intended to be within the scope of the claims appended hereto.

1. A triple co-culture composition comprising:
a first cell, wherein the first cell is a patient-derived cancer cell;
a second cell, wherein the second cell is a non-tumor derived cell that supports growth and/or viability of the first cell; and
a third cell, wherein the third cell is a tumor-derived cell that is not derived from the patient and that exhibits sensitivity to at least one anti-cancer drug or drug combination.

2. The triple co-culture composition of claim 1, wherein the first cell and the third cell are derived from the same type of cancer.

3. The triple co-culture composition of claim 1, wherein the second cell and the third cell are non-isogenic.

4. The triple co-culture composition of claim 1, wherein the first cell, second cell, and the third cell are human cells.

5. The triple co-culture composition of claim 1, wherein the second cell is of a non-tumoral cell type that is present in a tumor microenvironment of the first cell.

6. The triple co-culture composition of claim 5, wherein the second cell is a bone marrow stromal cell and the first cell is a patient-derived cancer cell of hematological origin.

7. The triple co-culture of claim 5, wherein the second cell is a fibroblast and the first cell is a patient-derived cancer cell from a solid tumor.

8. The triple co-culture composition of claim 1, wherein at least one of the second cell and the third cell comprises a heterologous gene.

9. The triple co-culture composition of claim 8, wherein the second cell is labeled with a first reporter.

10. The triple co-culture composition claim 9, wherein the third cell is labeled with a second reporter.

11. The triple co-culture composition of claim 10, wherein the first reporter and the second reporter are different from each other.

12. The triple co-culture composition of claim 11, wherein at least one of the first reporter and the second reporter is a fluorescent protein.

13. The triple co-culture composition of claim 1, wherein the second cell and third cell promote growth of the first cell in the triple co-culture.

14. The triple co-culture composition of claim 1, wherein the first cell is a blood cancer cell.

15. The triple co-culture composition of claim 14, wherein the blood cancer is a leukemia.

16. The triple co-culture composition of claim 1, wherein the first cell is a cell from a solid tumor.

17. The triple co-culture composition of claim 1, wherein the first cell, second cell, and third cell are in a cellular aggregate.

18. The triple co-culture composition of claim 17, wherein the first cell, second cell, and third cell in the cellular aggregate adhere to each other via extracellular matrix proteins.

19. The triple co-culture composition of claim 17, wherein the composition further comprises a patient derived non-cancer cell from the patient's cancer microenvironment.

20. The triple co-culture composition of claim 19, wherein the patient derived non-cancer cell from the patient's cancer microenvironment is a healthy stromal cell, an infiltrating immune cell, or an endothelial cell.

21. The triple co-culture composition of claim 1, wherein:
i) when the first cell is a patient derived cancer cell of a certain cancer of a hematological origin; the second cell is a bone marrow stromal cell; and the third cell is a cancer cell of the certain cancer of the hematological origin; or
ii) when the first cell is a patient derived cell of a solid tumor, the second cell is a fibroblast, vascular endothelial cell, pericyte, or adipocyte, and the third cell is a cancer cell of the solid tumor.

22. The triple co-culture composition of claim 21, wherein:
i) when the first cell is a patient derived cancer cell of a certain cancer of the hematological origin selected from leukemia, lymphoma, and myeloma;
ii) the second cell is a bone marrow stromal cell; and
iii) the third cell is a cancer cell of the certain cancer of the hematological origin.

23. The triple co-culture composition of claim 22, wherein:
i) when the first cell is a patient derived leukemia cancer cell; the second cell is a bone marrow stromal cell; and the third cell is leukemia cancer cell;
ii) when the first cell is a patient derived lymphoma cell; the second cell is a bone marrow stromal cell; and the third cell is a lymphoma cell; or
iii) when the first cell is a patient derived myeloma cell; the second cell is a bone marrow stromal cell; and the third cell is a myeloma cell.

24. The triple co-culture composition of claim 21, wherein:
the first cell is a patient derived cancer cell of a certain solid tumor selected from acral lentiginous melanoma, adenocarcinoma, adenoma, anaplastic thyroid cancer, brain tumor, breast cancer, colon cancer, colorectal cancer, cutaneous T-cell lymphoma, epithelioid sarcoma, esophageal cancer, follicular lymphoma, gastrointestinal cancer, head and neck cancer, hepatocellular carcinoma, intraocular melanoma, melanoma, nodular melanoma, nonmelanoma skin cancer, non-small cell lung cancer, ovarian cancer, ovarian epithelial cancer, pancreatic cancer, and uveal melanoma; the second cell is a fibroblast, vascular endothelial cell, pericyte, or adipocyte; and the third cell is a cancer cell of the certain solid tumor.

25. The triple co-culture composition of claim 21, wherein:
i) when the first cell is a patient derived acral lentiginous melanoma cell; the second cell is a fibroblast, vascular endothelial cell, pericyte, or adipocyte; and the third cell is an acral lentiginous melanoma cell;
ii) when the first cell is a patient derived adenocarcinoma cell; the second cell is a fibroblast, vascular endothelial cell, pericyte, or adipocyte; and the third cell is an adenocarcinoma cell;
iii) when the first cell is a patient derived adenoma cell; the second cell is a fibroblast, vascular endothelial cell, pericyte, or adipocyte; and the third cell is an adenoma cell;
iv) when the first cell is a patient derived thyroid cancer cell; the second cell is a fibroblast, vascular endothelial cell, pericyte, or adipocyte; and the third cell is a thyroid cancer cell;
v) when the first cell is a patient derived brain cancer cell; the second cell is a fibroblast, vascular endothelial cell, pericyte, or adipocyte; and the third cell is a brain cancer cell;
vi) when the first cell is a patient derived breast cancer cell; the second cell is a fibroblast, vascular endothelial cell, pericyte, or adipocyte; and the third cell is a breast cancer cell;
vii) when the first cell is a patient derived colon cancer cell; the second cell is a fibroblast, vascular endothelial cell, pericyte, or adipocyte; and the third cell is a colon cancer cell;
viii) when the first cell is a patient derived colorectal cancer cell; the second cell is a fibroblast, vascular endothelial cell, pericyte, or adipocyte; and the third cell is a colorectal cancer cell;
ix) when the first cell is a patient derived cutaneous T-cell lymphoma cell; the second cell is a fibroblast, vascular endothelial cell, pericyte, or adipocyte; and the third cell is a cutaneous T-cell lymphoma cell;
x) when the first cell is a patient derived epithelial sarcoma cell; the second cell is a fibroblast, vascular endothelial cell, pericyte, or adipocyte; and the third cell is an epithelial sarcoma cell;
xi) when the first cell is a patient derived esophageal cancer cell; the second cell is a fibroblast, vascular endothelial cell, pericyte, or adipocyte; and the third cell is an esophageal cancer cell;
xii) when the first cell is a patient derived follicular lymphoma cell; the second cell is a fibroblast, vascular endothelial cell, pericyte, or adipocyte; and the third cell is a follicular lymphoma cell;
xiii) when the first cell is a patient derived gastrointestinal cancer cell; the second cell is a fibroblast, vascular endothelial cell, pericyte, or adipocyte; and the third cell is a gastrointestinal cancer cell;
xiv) when the first cell is a patient derived head and neck cancer cell; the second cell is a fibroblast, vascular endothelial cell, pericyte, or adipocyte; and the third cell is a head and neck cancer cell;
xv) when the first cell is a patient derived hepatocellular carcinoma cell; the second cell is a fibroblast, vascular endothelial cell, pericyte, or adipocyte; and the third cell is a hepatocellular carcinoma cell;
xvi) when the first cell is a patient derived intraocular melanoma cell; the second cell is a fibroblast, vascular endothelial cell, pericyte, or adipocyte; and the third cell is an intraocular melanoma cell;
xvii) when the first cell is a patient derived melanoma cell; the second cell is a fibroblast, vascular endothelial cell, pericyte, or adipocyte; and the third cell is a melanoma cell;
xviii) when the first cell is a patient derived nodular melanoma cell; the second cell is a fibroblast, vascular endothelial cell, pericyte, or adipocyte; and the third cell is a nodular melanoma cell;
xix) when the first cell is a patient derived nonmelanoma skin cancer cell; the second cell is a fibroblast, vascular endothelial cell, pericyte, or adipocyte; and the third cell is a nonmelanoma skin cancer cell;
xx) when the first cell is a patient derived non-small cell lung cancer cell; the second cell is a fibroblast, vascular endothelial cell, pericyte, or adipocyte; and the third cell is a non-small cell lung cancer cell;
xxi) when the first cell is a patient derived ovarian cancer cell; the second cell is a fibroblast, vascular endothelial cell, pericyte, or adipocyte; and the third cell is an ovarian cancer cell;
xxii) when the first cell is a patient derived ovarian epithelial cancer cell; the second cell is a fibroblast, vascular endothelial cell, pericyte, or adipocyte; and the third cell is an ovarian epithelial cancer cell;
xxiii) when the first cell is a patient derived pancreatic cancer cell; the second cell is a fibroblast, vascular endothelial cell, pericyte, or adipocyte; and the third cell is a pancreatic cancer cell;
xxiv) when the first cell is a patient derived uveal melanoma cell; the second cell is a fibroblast, vascular endothelial cell, pericyte, or adipocyte; and the third cell is a uveal melanoma cell.

26. A method for detecting a response of a patient-derived cancer cell to an anti-cancer therapeutic comprising:
culturing the triple co-culture according to claim 1 in the presence of a test agent, wherein the test agent is an anti-cancer therapeutic or a candidate anti-cancer therapeutic; and
detecting the response to the test agent of the first cell, the second cell, and the third cell.

27. The method of claim 26, comprising:
culturing the triple co-culture for a first time period;
administering to the co-culture the test agent at the conclusion of the first time period; and detecting the response to the test agent, of the first cell, the second cell, and the third cell after a second time period.

28. The method of claim 27, further comprising comparing the response to the test agent of the first cell to the response to the test agent of the second cell and the third cell.

29. The method of claim 26, wherein the response comprises changes in cell growth, cell number, cell size, and/or cell morphology.

30. The method of claim 27, wherein the second period comprises at least one cell cycle of the first cell.

31. The method of claim 26, further comprising scoring the first cell as sensitive to the test agent if the first cell exhibits an adverse response after the second time period, and the second cell does not exhibit an adverse response after the second time period.

32. The method of claim 31, further comprising scoring the first cell as sensitive if the third cell exhibits an adverse response after the second time period.

33. The method of claim 31, wherein the adverse response comprises apoptosis, necrosis, an inhibition of cell growth, an inhibition of cell division, or an abnormal cell morphology.

34. The method of any of claim 32, wherein the second cell comprises a first reporter and the third cell comprises a second reporter, and wherein detecting the adverse response comprises detecting a loss of reporter activity.

35. The method of claim 26, wherein detecting the response to the test agent comprises fluorescent activated cell sorting (FACS).

36. The method of claim 27, wherein detecting the response to the test agent comprises FACS at the conclusion of the second time period.

37. A method for analyzing a set of test agents comprising:
culturing a set of triple co-cultures, wherein each triple co-culture of the set comprises the triple co-culture according to claim 1, and wherein each triple co-culture in the set comprises the same patient derived cancer cell, and the same second cell and third cell;
administering to each triple co-culture of the set a different test agent from the set of test agents or a different concentration of the same test agent; and
detecting a response to the test agents of the first cell, the second cell, and the third cell in each triple co-culture.

38. The method of claim 37, comprising:
culturing each triple co-culture for a first time period;
at the conclusion of the first time period, administering to each co-culture a test agent from the set of test agents or a different concentration of the same test agent; and
detecting the response to the test agent of the first cell, the second cell and the third cell in each co-culture after a second time period.

39. The method of claim 37, further comprising comparing the response to the test agent of the first cell to the response to the test agent of the second cell and the third cell.

40. The method of claim 37, wherein the response comprises changes in cell growth, cell number, cell size, and/or cell morphology.

41. The method of claim 38, wherein the second period comprises at least one cell cycle of the first cell.

42. The method of claim 38, further comprising scoring the first cell as sensitive to the test agent if the first cell exhibits an adverse response after the second time period, and the second cell does not exhibit an adverse response after the second time period.

43. The method of claim 42, further comprising scoring the first cell as sensitive if the third cell exhibits an adverse response after the second time period.

44. The method of claim 42, wherein the adverse response comprises apoptosis, necrosis, an inhibition of cell growth, an inhibition of cell division, or an abnormal cell morphology.

45. The method of claim 38, wherein the second cell comprises a first reporter and the third cell comprises a second reporter and detecting the adverse response comprises detecting a loss of reporter activity.

46. The method of claim 37, wherein detecting the response to the test agent comprises FACS.

47. The method of claim 37, wherein detecting the response to the test agent comprises FACS at the conclusion of the second time period.

48. A method for preparing a triple co-culture composition comprising:
a first cell, wherein the first cell is a patient-derived cancer cell;
a second cell, wherein the second cell is a non-tumor derived cell that supports growth and/or viability of the first cell; and
a third cell, wherein the third cell is a tumor-derived cell that is not derived from the patient and that exhibits sensitivity to at least one anti-cancer drug or drug combination, wherein the first cell, second cell, and third cell are in a cellular aggregate;
the method comprising mixing the first cell, the second cell, and the third cell and facilitating the formation of the cellular aggregate.

49. The method of claim 48, wherein facilitating the formation of the cellular aggregate comprises incubating the first cell, the second cell, and the third cell with biologically functionalized magnetic nanoparticles that render the cells magnetic and applying an external magnetic field to assemble the magnetic first cell, the second cell, and the third cell into the cellular aggregate.

50. The method of claim 48, wherein facilitating the formation of the cellular aggregate comprises applying the first cell, the second cell, and the third cell into a container having a cell-repellent inner surface, optionally, centrifuging the container, and incubating the cells for a sufficient period to allow the cells added to the container to coalesce into the cellular aggregate.

51. The method of claim 48, comprising mixing the first cell, the second cell, and the third cell with a patient derived non-cancer cell from the patient's cancer microenvironment.

52. The method of claim 51, wherein the patient derived non-cancer cell from the patient's cancer microenvironment is a healthy stromal cell, an infiltrating immune cell, or an endothelial cell.

53. The method of claim 26, wherein the method is performed in vitro.
